(19) United States
(12) Patent Application Publication
(10) Pub. No.: US 2012/0244593 A1
(43) Pub. Date: Sep. 27, 2012

(54) METHOD, DEVICE AND APPARATUS FOR INDUCING SELF-ADJUSTING CELL ELECTROPORATION

(76) Inventors: Yong Huang, Milpitas, CA (US); Xupeng Xi, Fremont, CA (US)

(21) Appl. No.: 13/502,334

(22) PCT Filed: Oct. 19, 2010
(86) PCT No.: PCT/US10/53279

§ 371 Date: Jun. 7, 2012

Related U.S. Application Data
(60) Provisional application No. 61/253,050, filed on Oct. 19, 2009.

(51) Int. Cl. C12N 13/00; C12M 1/42

(52) U.S. Cl. 435/173.6; 435/285.2

(57) ABSTRACT

The invention provides methods, devices and apparatuses for providing cell electroporation. In accordance with an aspect of the invention, self-adjusting cell electroporation may be provided. A cell transfection apparatus may be provided. The cell transfection apparatus may include a plate comprising a plurality of chambers configured to receive and confine a population of host cells. The cell transfection apparatus may also include a plurality of electrodes configured to be in electrical communication with a corresponding chamber of said plate. A predetermined electrical current may be directed through a chamber to effect transfection of said host cells.

### CROSS-REFERENCE

This application claims the benefit of U.S. Provisional Application No. 61/253,050, filed Oct. 19, 2009, which application is incorporated herein by reference in its entirety for all purposes.

### STATEMENT AS TO FEDERALLY SPONSORED RESEARCH

This invention was made with government support under RR022955 awarded by National Center for Research Resources/NIH. The government has certain rights in the invention.

### BACKGROUND OF THE INVENTION

Electroporation is a process associated with transient permeabilization of cell membranes under electrical fields. It has been shown to be capable of delivering various macromolecules (e.g., genes, siRNAs, antibodies and proteins) into virtually any type of cells (e.g., cell lines and primary cells). Traditionally, electroporation is performed with cells in suspension, with hundreds of volts of short pulses applied to the cells between two electrodes. Whether a cell can be electroporated is determined by electrical potential drop across the cell, which depends on cell size and other factors. A cell population typically has large variation in the size of individual cells.

As a result, using the traditional approach, small cells often do not have sufficient cross-membrane potential, and are not electroporated. On the other hand, large cells can experience high cross-membrane electrical potential, which results in irreversible electroporation or membrane disruption that causes cell death. Therefore, the traditional electroporation approach inevitably results in substantial variations in electroporation effectiveness and causes cell death in large portion of cells, because of heterogeneity in cell size and state affecting membrane electropermeabilization.

Thus, a need exists for improved methods and systems of cell electroporation that may accommodate larger variation in cell size and state.

### SUMMARY OF THE INVENTION

An aspect of the invention is directed to a cell transfection apparatus. The cell transfection apparatus may comprise (a) a plate having a plurality of chambers, each configured to receive and confine a population of host cells, (b) a plurality of electrodes, each of which being configured to be in electrical communication with a corresponding chamber of said plate, (c) an input unit adapted to receive input of one or more transfection parameters from a user, and (d) a controller for processing said input and effecting an electrical current for a defined period of time to at least one electrode of said plurality, based on the input of said one or more transfection parameters from said user.

Another aspect of the invention is directed to a method for transfecting a population of host cells. The method may comprise the steps of (a) providing a cell transfection apparatus as described elsewhere, (b) receiving input of one or more transfection parameters from a user, and (c) processing said input and effecting an electrical current for a defined period of time to said population of host cells, based on the input of said one or more transfection parameters from said user.

A method for transfecting a population of host cells may be provided in accordance with another embodiment of the invention. The method may include the steps of providing a cell transfection apparatus, including those described elsewhere herein, wherein the apparatus comprises a touch screen having a visual representation of the plate having a plurality of chambers; receiving a user input through the touch screen, wherein the input defines a transfection parameter relating to at least one chamber of said plate; and processing said input and effecting an electrical current for a defined period of time to said chamber, based on the input of said transfection parameter from said user.

In accordance with another aspect of the invention, a method of selecting a transfection condition may be provided. The method of selecting a condition may comprise (a) providing a plate having a plurality of chambers, wherein at least one individual chamber of said plurality contains a population of host cells; (b) providing a plurality of electrodes, each of which being configured to be in electrical communication with a corresponding chamber of said plate; (c) measuring electrical resistance of the contents of said at least one individual chamber and determining a desired transfection condition to effect said transfection.

A cell transfection device may be provided in accordance with an aspect of the invention. The cell transfection device may include an electrode assembly, and a plate holder configured to hold a transfection plate, wherein the plate holder is configured to translate the plate into alignment with the electrode assembly, wherein the plate holder and/or the electrode assembly is movable in an upward or downward direction so that the electrode assembly and the transfection plate or contents thereof are brought into contact with one another.

A cell transfection device may be provided in accordance with an aspect of the invention. The cell transfection device may include an electrical connection assembly, and a plate holder configured to hold a transfection assembly, the transfection assembly comprising a transfection plate and an electrode plate, wherein the plate holder is configured to translate the transfection assembly into alignment with the electrical connection assembly, wherein the plate holder and/or the electrical connection assembly is movable in an upward or downward direction so that the electrical connection assembly and the transfection assembly or contents thereof are brought into contact with one another.

A display device showing a graphical user interface may be provided comprising a visual representation of a cell transfection plate having a plurality of chambers, wherein said visual representation includes images corresponding to said plurality of chambers. The display device may also include a user interactive control that permits a user to define a property relating to at least one chamber via the visual representation.

A method for transfecting a foreign substance into a population of host cells may be provided in accordance with another aspect of the invention. The method may comprise the step of (i) providing a cell transfection apparatus comprising: (a) a plate comprising a plurality of chambers, each of said plurality of chambers being configured to receive and confine a population of host cells; and (b) a plurality of electrodes, each of which being configured to be in electrical communication with a corresponding chamber of said plate. The method may also include the step of (ii) directing a predetermined electrical current through at least said corresponding chamber to effect transfection of said host cells.

Additional aspects and advantages of the present disclosure will become readily apparent to those skilled in this art from the following detailed description, wherein only exemplary embodiments of the present disclosure are shown and described, simply by way of illustration of the best mode contemplated for carrying out the present disclosure. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 provides a configuration for traditional electroporation using voltage pulses.

FIG. 2 shows biological cells that are attached to a substrate in accordance with an embodiment of the invention.

FIG. 3 shows a simplified electrical circuit model in accordance with an embodiment of the invention.

FIG. 4 shows an example of a method for selecting a transfection condition in accordance with an embodiment of the invention.

FIG. 5 shows an example of an electroporation process accounting for feedback measurements.

FIG. 6 provides an example of an error state that may be determined.

FIG. 7 shows an example of a typical electrical current pulse for inducing electroporation.

FIG. 8A shows an example of a substrate functioning as an electrode and support for cells in accordance with an embodiment of the invention.

FIG. 8B shows an example of a porous substrate membrane in accordance with another embodiment of the invention.

FIG. 9A shows an example of providing a unidirectional current to cells on a substrate in a first direction.

FIG. 9B shows an example of providing a unidirectional current to cells on a substrate in a second direction.

FIG. 10 shows a configuration of a cell electroporation device with cells attached to an electrode in accordance with an embodiment of the invention.

FIG. 11 shows a configuration of a cell electroporation device with cells attached to a substrate that allows current passage in accordance with another embodiment of the invention.

FIG. 12 shows a configuration of a cell electroporation device with a common bottom electrode and a common bottom/basal chamber in accordance with an embodiment of the invention.

FIG. 13 shows an example of a unidirectional current used within a cell electroporation device that may assist with preventing cross-contamination.

FIG. 14 provides an illustration of using pressure to position cells on specific locations of a porous membrane.

FIG. 15 shows an example of a cell electroporation device in a microtiter plate format.

FIG. 16A-C shows views of an embodiment of the cell electroporation device.

FIG. 17A-D shows views of another embodiment of the cell electroporation device.

FIG. 18 shows an example of an electrode configuration.

FIG. 19 shows a design of a cell electroporation device for transfecting cells in microtiter plate format

FIG. 20A shows an example where an electrode is brought into electrical communication with contents of a chamber through the top.

FIG. 20B shows an example where an electrode is brought into electrical communication with contents of a chamber through the bottom.

FIG. 21 shows a depiction of a transfection plate being translated into alignment with an electrode assembly.

FIG. 22A shows an example of a transfection plate and an electrode assembly being brought into contact with one another.

FIG. 22B shows another example of a transfection plate and an electrode assembly being brought into contact with one another.

FIG. 23 shows an example of a configuration of a cell electroporation apparatus.

FIG. 24 shows an example of a cell electroporation system in accordance with an embodiment of the invention.

FIG. 25 shows an example of a touch-screen based GUI for inducing cell electroporation in a 96-well format in accordance with an embodiment of the invention.

FIG. 26 shows an example of a user interface display providing main options to a user.

FIG. 27 shows a user interface used for transfection.

FIG. 28 shows a user interface displaying a transfection status after electroporation.

FIG. 29 shows an example of an optimization graphical user interface.

FIG. 30 shows a system suggested protocol based on a detected electrical property of a cell layer.

FIG. 31 provides an example of a user interface where a user may further edit a protocol.

FIG. 32 shows how a plurality of different protocols can be applied to one plate.

FIG. 33 shows a user interface which allows users to manage their own protocols.

FIG. 34 shows a screen to allow a user to create or edit existing protocols.

FIG. 35 shows a screen where the system can diagnose whether it is working properly.

FIG. 36 shows a display after the diagnostics have been completed.

### DETAILED DESCRIPTION OF THE INVENTION

While preferable embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

The invention provides methods, devices and apparatuses for providing cell electroporation. Various aspects of the invention described herein may be applied to any of the particular applications set forth below or for any other types of cell electroporation or data management. The invention may be applied as a standalone tool or as part of an integrated platform used to perform and/or control cell electroporation. Some preferable embodiments of the invention can include self-adjusting cell electroporation with a predetermined current. It shall be understood that different aspects of the invention can be appreciated individually, collectively or in combination with each other.

Electroporation is a process associated with transient permeabilization of cell membranes under electrical fields. It has been shown to be capable of delivering various macromolecules (e.g., genes, siRNAs, antibodies and proteins) into virtually any type of cells (e.g., cell lines and primary cells).

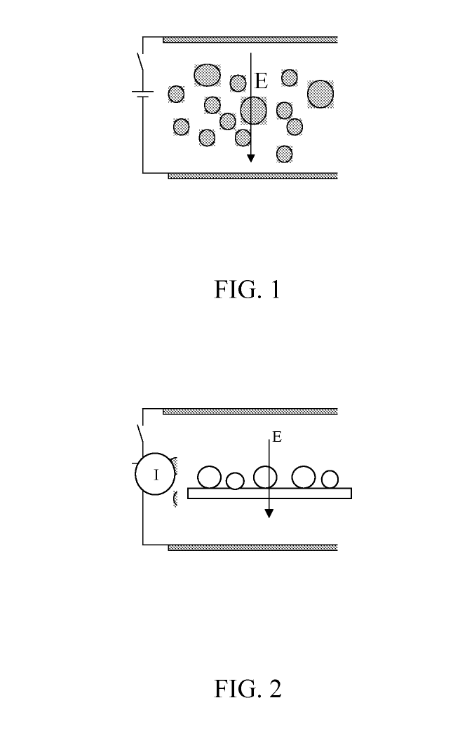

FIG. 1 provides a configuration for traditional electroporation using voltage pulses. Electroporation is typically done with cells in suspension, with hundreds of volts of short pulses applied to the cells between two electrodes. The electrodes may be used to apply the voltage to the heterogeneous population of cells in suspension. Whether a cell can be electroporated is determined by electrical potential drop across the cell, which is dependent on cell size and other factors. A cell population typically has large variation in the size of individual cells. As a result, using this simple approach, oftentimes small cells do not have sufficient cross-membrane potential, and therefore, are not electroporated. On the other hand, large cells can experience high cross-membrane electrical potential, which results in irreversible electroporation or membrane disruption that causes cell death. Therefore, the traditional electroporation approach inevitably results in substantial variations in electroporation effectiveness and causes cell death in large portion of cells, because of heterogeneity in cell size and state affecting membrane electropermeabilization.

I. Electroporation of Cells Attached on a Solid Surface Using Electrical Currents

One aspect of the invention provides a method of using electrical currents to electroporate biological cells. The biological cells may include primary cells, engineered cells, and/or cell lines. Preferably, the cells may be attached on a substrate that allows the electrical currents to pass through. Alternatively, the cells may be provided in suspension.

As illustrated in FIG. 2, biological cells are attached to a substrate that allows electrical current to pass through. Electrodes are placed on both sides of the substrate, and are connected to an electrical current source. If the substrate is made of electrical conductive material, it itself can serve as the bottom electrode (or alternatively, the top electrode depending on direction of current flow). In some embodiments, the electrodes may be silver/silver chloride (Ag/AgCl) electrodes. Other examples of electrodes may include but are not limited to metals such as copper, aluminum, gold, silver, platinum, or nickel, or any non-metallic conductors, or any alloys or combinations thereof. Any of the embodiments discussed elsewhere herein may incorporate cells attached to a substrate. Alternatively, any of the embodiments discussed elsewhere herein may include cells provided in suspension, or any combination of suspension or adherence.

When electrical current, I, is applied between the electrodes, there are three possible routes for the current to flow through the cell-substrate, i.e., I_{d} that is directly through area of the substrate that is not covered by cells, Iₚ that is through microscopic gaps between the substrate and biological cells (paracellular current), and Iₚ that is through biological cells. I is the sum of these three components. Since there is overall electrical resistance between the top and bottom of the cells, the overall electrical current I will induce a potential drop across the cells, V_{c}. When V_{c} is higher than a threshold electroporation potential (Vₑₚ), the biological cells undergo membrane permeabilization, which allows foreign substances (such as DNA, RNA, siRNA, microRNA, proteins, peptides, small molecules, nanoparticles, and other membrane impermeate molecules) to be delivered into the electroporated cells. The threshold electroporation potential Vₑₚ may depend on the type of biological cell undergoing membrane permeabilization.

In preferable embodiments, a predetermined electrical current, I, may be applied between the electrodes. In some embodiments, the electrical current may be slow-varying, substantially constant, or fixed during the duration of a cell electroporation process. Alternatively, the predetermined electrical current may vary during a cell electroporation procedure. The electrical current may be selected and controlled so that the electrical current is varied and/or maintained in a controlled manner. The predetermined electrical current may or may not be based on received signals.

One benefit of the current electroporation method lies in that in such configuration, biological cells adjust themselves in response to the electrical current, such that the cross-cell potential, V_{c}, is maintained at a “just right” value that is sufficient for electroporation but not high enough to trigger irreversible electroporation or membrane disruption, a phenomenon that is typically seen in electroporation when the cross-cell potential exceeds a threshold value for irreversible electroporation, Vᵢᵣₑ.

II. Self-Adjusting Cell Electroporation (SACE)

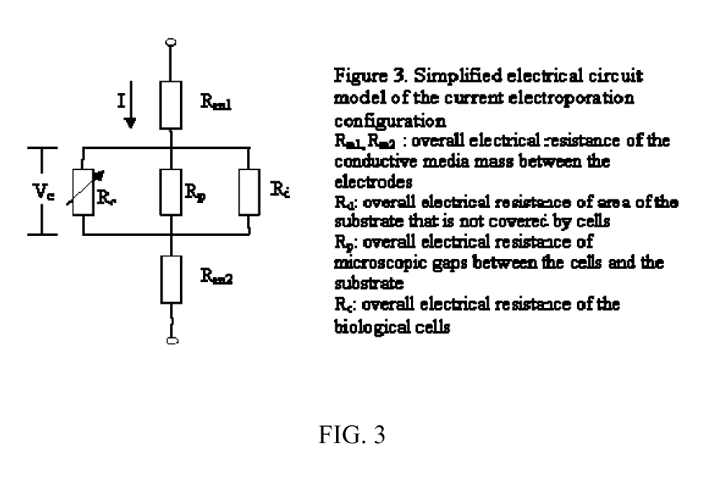

FIG. 3 shows a simplified electrical circuit model of the above-mentioned components between the electrodes. In some embodiments, a slow-varying electrical current may be used and capacitance of the components may be small, so that the effect of electrical impedance due capacitance of the components is negligible and thus omitted from FIG. 3.

Rₘ₁ and Rₘ₂ are electrical resistance of the conductive media mass between the electrodes, whose values are typically independent of cells. R_{d} is the overall electrical resistance of open area of the substrate not covered by cells, therefore, typically its value is also not dependent on cells. Rₚ is the overall resistance of the gaps between the cells and the substrate, its value depends on how tightly cells are attached to the substrate, but typically does not depends on cell membrane permeability.

R_{c} is the equivalent overall electrical resistance of the biological cells, which is determined by permeability of the cell membranes. Since electrical conductivity of an intact cell membrane is extremely low, intact cells typically have high R_{c}. When cells are effectively electroporated, the permeabilized cell membrane allows electrical currents to pass through, resulting in significantly reduced membrane electrical resistance. Since the degree of cell membrane permeabilization is determined by the electrical potential across the cells, i.e., V_{c}, R_{c} is also heavily dependent on V_{c}. On the other hand, V_{c} can be approximated as I×Rₐₗₗ, whereas Rₐₗₗ is the equivalent of R_{ce}, Rₚ and R_{d} in parallel, i.e., Rₐₗₗ=R_{c}//Rₚ//R_{d}.

In some embodiments, a continuous current may be provided. The continuous current may be maintained and/or varied. In some instances, the current may be substantially fixed or slow-varying. Alternatively, in preferable embodiments, a pulsed current may be provided.

In an embodiment of the invention, a precise electrical current I pulse, with pulse width ranging from milliseconds to seconds, may be applied between the electrodes through a current source. Any value of a current, I, may be used, including but not limited to currents falling between 0.1 mA and 100 A. This may include currents of about 1 mA, 5 mA, 10 mA, 50 mA, 100 mA, 500 mA, 1 A, 5 A, 10 A, 50 A, or 100 A. The current may be fixed during a pulse, or may vary. In some instances, the current may remain the same or be slow varying between multiple pulses, or may vary from pulse to pulse. In some instances, the current may remain the same or be slow varying throughout a cell transfection process, or may vary. The current value may be predetermined for the entire cell transfection process of a population of host cells, or may be adjusted based on feedback received during the process. A predetermined current amplitude may be applied based on one or more signals received before and/or during the electroporation process. The current value may be controlled throughout the cell electroporation.

The duration of the electrical pulses may also have any value, but not limited to pulses falling within 1 μs to 1 minute, 500 μs to 30 seconds, or 1 ms to 10 seconds. This may include pulse with a duration of about 1 μs, 5 μs, 10 μs, 50 μs, 100 μs, 500 μs, 1 ms, 3, ms, 5 ms, 10 ms, 50 ms, 100 ms, 250 ms, 500 ms, 750 ms, 1 second, 2 seconds, 3 seconds, 4 seconds, 5 seconds, 7 seconds, 10 seconds, 15 seconds, 20 seconds, 30 seconds, 45 seconds, or 1 minute. The duration of the pulses may remain fixed during a cell electroporation process, or may vary. The pulse duration may be predetermined for the entire cell transfection process of a population of host cells, or may be adjusted based on feedback received during the process. The pulse duration may be controlled throughout the cell electroporation.

The amount of time between the electrical pulses may also have any value, but not limited to time periods falling within 1 μs to 1 minute, 500 μs to 30 seconds, or 1 ms to 10 seconds. This may include time between pulses of about 1 μs, 5 μs, 10 μs, 50 μs, 100 μs, 500 μs, 1 ms, 3, ms, 5 ms, 10 ms, 50 ms, 100 ms, 250 ms, 500 ms, 750 ms, 1 second, 2 seconds, 3 seconds, 4 seconds, 5 seconds, 7 seconds, 10 seconds, 15 seconds, 20 seconds, 30 seconds, 45 seconds, or 1 minute. The time between pulses may be less than, the same as, or greater than the duration of the pulse. The time between the pulses may remain fixed during a cell electroporation process, or may vary. The time between pulses may be predetermined for the entire cell transfection process of a population of host cells, or may be adjusted based on feedback received during the process. The time between pulses may be controlled throughout the cell electroporation.

The number of electrical pulses and/or the number of cycles may also have any value. For example, a cycle may have one or more, two or more, three or more, four or more, five or more, six or more, seven or more, eight or more, nine or more, ten or more, twelve or more, fifteen or more, twenty or more, 30 or more, 50 or more, 70 or more, or 100 or more electrical pulses. Any number of cycles may be provided for an electroporation process. For example, one cycle may be provided for an electroporation process. Alternatively, two, three, four, five, six, seven, eight, nine, ten, or more cycles may be provided for an electroporation process. One or more cycles may be characterized by a break in electrical pulses that are provided. In another example, the various cycles may have different transfection conditions (e.g., different amplitudes, duration of pulses, etc.) from cycle to cycle. Alternatively, no difference needs to be provided between cycles.

In accordance with an aspect of the invention, a method for transfecting a foreign substance into a population of host cells may be provided. The method may comprise the steps of (i) providing a cell transfection apparatus comprising: (a) a plate comprising a plurality of chambers, each of said plurality of chambers being configured to receive and confine a population of host cells; and (b) a plurality of electrodes, each of which being configured to be in electrical communication with a corresponding chamber of said plate; and (ii) directing a predetermined electrical current through at least said corresponding chamber to effect transfection of said host cells.

In some embodiments, the predetermined electrical current is directed without controlling voltage across the chamber. The transfection may result in an uptake of a larger effective quantity of said foreign substance by said population of host cells than the uptake by said host cells via a transfection method utilizing predetermined voltage. In some embodiments, membranes of the host cells are kept open continuously for at least one millisecond during said transfection. The cell transfection apparatus may further comprise at least two sets of electrodes, wherein one of the sets is aligned on the top of the plate, and another set is aligned at the bottom of the plate, and wherein the two sets are individually controlled to provide a unidirectional current flowing from the top of the plate to the bottom of the plate or vice versa.

Without being bound by any particular theory, the following description outlines an example of a method and theory for transfecting a foreign substance into a host cell.

When the resulting V_{c} is sufficient to cause electroporation in the cells (V_{c}>Vₑₚ), the cell electrical resistance R_{c} decreases dramatically, resulting in a reduced Rₐₗₗ. When I is not changed or slow-varying, V_{c} is reduced accordingly, resulting in lesser degree of cell membrane electroporation. On the other hand, if cell membrane is lesser permeabilized, the R_{c} increases again, resulting in elevated V_{c} that leads to higher degree of membrane permeabilization.

As such, under this configuration, the biological cells can adjust their membrane permeability automatically in response to a properly chosen electrical current, resulting in a “just right” electrical potential across the cells, which can maintain the cells in permeabilized state for an extended period of time without causing irreversible electroporation or membrane disruption. This phenomenon may be referred to as Self-Adjusting Cell Electroporation (SACE). It should be noted that SACE can also be induced by applying a voltage pulse to the cells. SACE may advantageously be able to accommodate the electroporation of heterogeneity in cells, such as cell size, membrane characteristics, or type. SACE may offer improved electroporation over the traditional approach which delivers a controlled voltage to a heterogeneous population of cells.

As discussed above, in order for the cells to be electroporated but not damaged by irreversible electroporation, the cross-cell potential difference, V_{c} should be

Vₑₚ<V_{c}<Vᵢᵣₑ (Equation 1)

whereas Vₑₚ is the threshold potential corresponding to onset of reversible electroporation, and Vᵢᵣₑ is the threshold potential corresponding to irreversible electroporation; whereas

V_{c}=I×Rₐₗₗ=I×(R_{c}/R_{d}//Rₚ) (Equation 2)

The assumption is made that when the electrical potential across the cells is at Vₑₚ, the corresponding cell electrical resistance is R_{c}_{_{—}}ₑₚ; and that when the electrical potential across the cells is at Vᵢᵣₑ, the corresponding cell electrical resistance is R_{c}_{_{—}}ᵢᵣₑ. The range of applied electrical current, I, that result in effective SACE without cell damage, can be estimated as

I_{sace}<I<Iᵢᵣₑ (Equation 3)

whereas I_{sace} is the threshold current that can induce SACE, and Iᵢᵣₑ is the threshold current that can trigger irreversible electroporation.

I_{sace}=Vₑₚ/(R_{c}_{_{—}}ₑₚ//R_{d}/Rₚ) (Equation 4)

Iᵢᵣₑ=Vᵢᵣₑ/(R_{c}_{_{—}}ᵢᵣₑ//R_{d}/Rₚ) (Equation 5)

Thus, a current I to be provided for cell electroporation may be selected to fall between I_{sace} and Iᵢᵣₑ. Values of the parameters in the equation can be determined experimentally or estimated with theoretical modeling. In some embodiments, one or more parameters may be stored in a database or in any type of memory. Such parameters may be accessed based on cell type or any other characteristic associated with the host cells undergoing electroporation. In some embodiments, one or more parameter may be measured prior to effecting the cell electroporation.

According to these equations, it is clear that increased R_{d} and Rₚ will increase the sensitivity of V_{c} on R_{c}, or on the electroporation state of the cells. R_{d} can be increased by reducing the area of the substrate not covered by cells, i.e., by increasing confluence level of the attached cells. In other words, R_{d} can be increased by increasing cell density on the substrate. Rₚ is dependent on how well cells are attached to the substrate, therefore it is largely dependent on cell types as well as surface properties of the substrate. One way to increase Rₚ is to conduct surface treatment on the substrate to increase cell-substrate attachment, such surface treatment include plasma based tissue culture treatment, coating with polylysine and extracellular matrix proteins such as fibronectin, collagen, etc.

Various transfection conditions may be provided during SACE. Some examples of transfection conditions may include cell type, membrane resistance, voltage, duration of current flow, rate of current flow, amplitude of current flow, number of electric pulses, shape of electrical pulses, and choice of transfection plate. The transfection conditions may affect the current, I, provided during transfection. Transfection conditions may affect other aspects of the SACE, such as voltage. Any discussion herein of SACE may also be applied to any form of cell electroporation. Any discussion elsewhere of cell transfection or cell electroporation may also be applied to SACE.

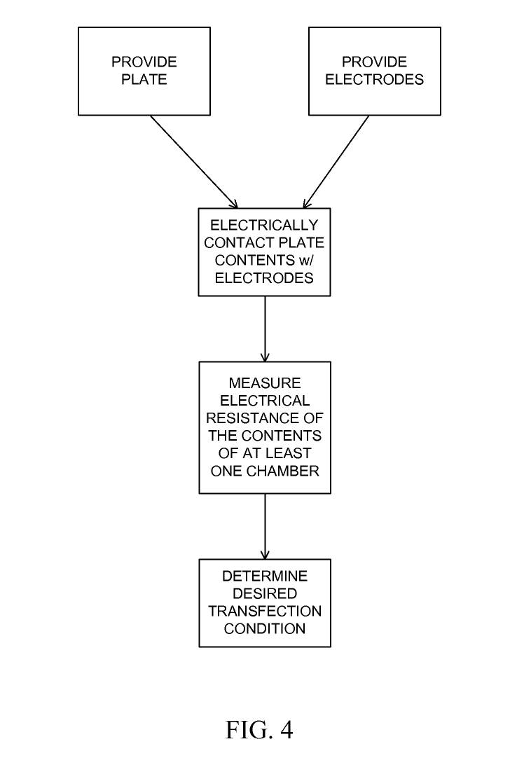

FIG. 4 shows an example of a method for selecting a transfection condition in accordance with an embodiment of the invention. The method may include providing a cell transfection device, which may have at least one chamber configured to contain a population of host cells. The method may also include providing one or more electrodes, which may be configured to be in electrical communication with the corresponding chamber and/or contents therein. In some embodiments, the transfection device may be a plate with a plurality of chambers, to be discussed in greater detail elsewhere herein. In some embodiments, the electrode may be brought into electrical contact with the corresponding chamber.

The method may also include the step of measuring an electrical property of the contents of at least one chamber. The electrical property may preferably be electrical resistance. Other examples of electrical properties may include electrical conductivity, electrical impedance, electrical reactance, voltage, magnetic flux or field, electrical charge, or electrical field or power. A predetermined transfection condition may be selected to effect cell transfection of the host cells. The predetermined transfection condition may be selected based on the measured electrical property (e.g., electrical resistance). Some examples of transfection conditions may include cell type, membrane resistance, voltage, duration of current flow, rate of current flow, amplitude of current flow, number of electric pulses, shape of electrical pulses, and choice of transfection plate. In some instances, one, two, or more transfection conditions may be selected. Each transfection condition may be selected based on the measured electrical property, or other selected transfection conditions. Thus, the various transfection conditions associated with a current may be selected, resulting in a predetermined current, which may be delivered to the contents of a chamber.

The method may be provided wherein the plurality of chambers are arranged as an array. In some instances, the plate may have a 6, 24, 48, 96, 384, or 1536 well format, as described elsewhere herein. In some instances, the host cells may be selected from the group consisting of: engineered cells, cell lines, and primary cells. Furthermore, an individual chamber may contain a population of host cells with density falling between 500 and 1,000,000 cells per square centimeter.

In accordance with an aspect of the invention, once a transfection condition has been selected, it may remain fixed throughout the duration of a cell electroporation process. A cell electroporation process may vary in duration and may depend on an estimated amount of time for cell electroporation to be complete. In some embodiments, the cell electroporation process may be selected by a user, automatically determined by a program, or may depend on measurements that may be taken during the cell electroporation process. In some instances, a cell transfection condition may be varied and/or maintained during the cell electroporation process. The cell transfection conditions may be varied and/or maintained in a predetermined manner. The conditions may or may not be altered based on subsequent signals or instructions during the cell electroporation process. A cell electroporation process may encompass one, or a plurality of cycles.

The cell transfection condition may be controlled. In some embodiments, the predetermined cell transfection condition may be controlled by a user input during the cell electroporation process. In other embodiments, the cell transfection current may follow a protocol that may have been selected or determined prior to or at the beginning of the cell electroporation process. The protocol may cause the transfection current to be varied or maintained in a predetermined manner. In some instances, the protocol selected at the beginning may be followed without any feedback. Alternatively, it may be automatically controlled in response to measurements taken during the cell electroporation process.

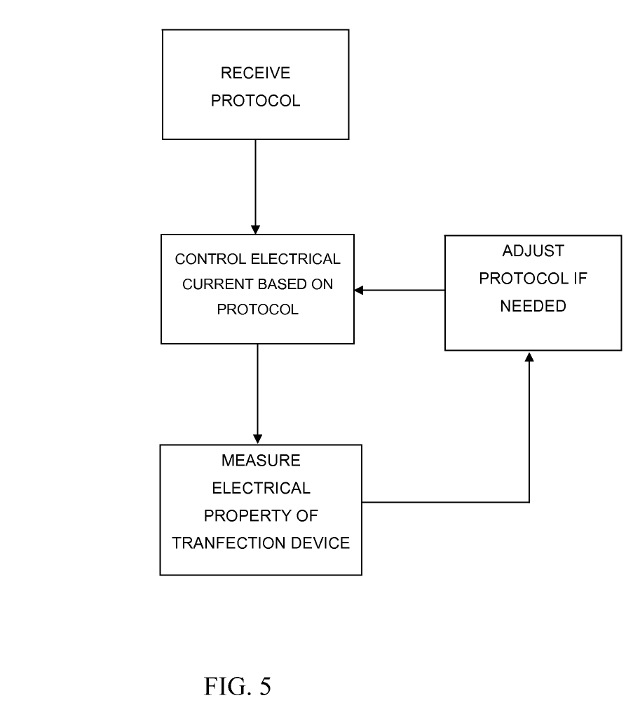

FIG. 5 shows an example of an electroporation process accounting for feedback measurements. In accordance with one method of controlling a cell electroporation process, the electrical resistance (or any other electrical property) of a transfection device contents may be measured. Based on said measurement, one or more transfection condition may be selected. A current may be delivered to the transfection device contents in accordance with the selected transfection conditions. A subsequent measurement of the electrical resistance (or any other electrical property) may be made. Based on the subsequent measurement, one or more transfection condition may be varied or maintained.

In some embodiments, an error state may be determined before or during cell transfection. For example, an electrical property may be measured, and based on said measurement an error state may be determined.

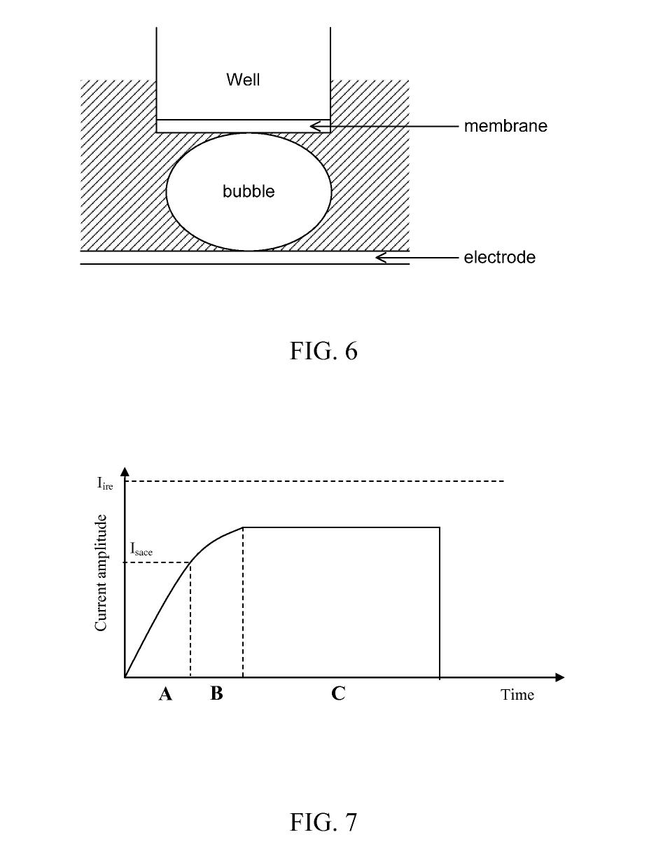

FIG. 6 provides an example of an error state that may be determined. A bubble may be trapped between a cell substrate and an electrode, or between a cell layer on a substrate and an electrode. The resistance of the contents of a chamber may be measured. If a bubble exists, the resistance measurement may be higher than expected. If the resistance measured exceeds a predetermined threshold resistance, an alert may be provided that an error has occurred. The resistance measurement may be taken prior to providing a current for the cell electroporation process. Alternatively, the resistance or any other electrical property may be measured during the cell electroporation process.

In some embodiments, if an error is determined, a cell electroporation process may be modified, or stopped. Alternatively, the cell electroporation process may continue. If an error is detected for a specific chamber of a transfection device, the electroporation process may or may not continue for the rest of the chambers of the transfection device.

In some embodiments, a user may be able to select a diagnosis option to test a transfection device, such as a transfection plate. One or more different types of errors may be detected using the diagnostic process. For example, a bubble may be detected between a cell substrate and an electrode. In another example, cell confluence level may be detected. For example, more efficient or effective electroporation may be provided when the cells are fully confluent, or covering the substrate. If gaps are provided between cells, current leakage may occur. In some instances, if the cells are not sufficiently confluent, the diagnosis option may return the result. An error detection system may return whether any condition is provided that may result in ineffective or inefficient electroporation.

In a typical experiment to induce cell electroporation (which may include SACE) in cells with electrical current, cells can undergo one or a combination of the following three phases. Initially, electrical current is gradually increased and before its amplitude reaches the threshold SACE current, I_{sace}, cell membrane does not undergo effective electroporation, which may be called Intact Phase (Phase A). After I exceeds I_{sace}, cell electroporation may happen and the degree of membrane permeability continue to increase as the current increases, which may be called Opening Phase (Phase B). At last, the electrical current may be kept at a constant value, and correspondingly, cell membrane permeabilization will experience a dynamic self-adjusting process to maintain membrane permeability at a certain degree. This may be referred to as a Maintenance Phase (Phase C). To avoid irreversible electroporation, the electrical current should not exceed Iᵢᵣₑ defined in Equation 5.

FIG. 7 shows an example of a typical electrical current pulse for inducing cell electroporation (e.g., SACE). A given pulse may undergo an Intact Phase, Opening Phase, and Maintenance Phase. Alternatively, it may undergo one or more of any of these phases. In some instances, a plurality of electrical pulses may be provided during a cell electroporation process. Each electrical pulse may undergo the same phases, or may have different current amplitude values. In some instances, a plurality of pulses may be provided that may follow the overall phases.

An electrical current pulse may start with an increasing current amplitude that may level out. The current level may be varied or maintained in any manner for a pulse. A current level may be increased, decreased, or maintained at a constant level during a pulse.

One of the advantages of current electroporation is that cell membranes can be kept open for a relatively long period of time (from milliseconds to seconds), compared to microseconds with traditional electroporation. In some embodiments, during cell transfection, the cell membrane may be kept open for any length of time, including microseconds. In preferable embodiments, the cell membrane may be kept open for at least 1 ms or longer, 3 ms or longer, 5 ms or longer, 10 ms or longer, 15 ms or longer, 20 ms or longer, 25 ms or longer, 50 ms or longer, 100 ms or longer, 150 ms or longer, 200 ms or longer, 300 ms or longer, 400 ms or longer, 500 ms or longer, 700 ms or longer, 1 second or longer, 1.5 seconds or longer, 2 seconds or longer, 3 seconds or longer, 4 seconds or longer, 5 seconds or longer, 7 seconds or longer, 10 seconds or longer. As result, more molecules can be introduced into the electroporated cells.

For molecules that carry net charges, such as nucleic acids (DNA, RNA, siRNA, microRNA, etc.) that are negatively charged, the electrical field can either facilitate or hinder delivery of the charge molecules into the electroporated cells, depending on where the molecules are brought to the cells and the polarity of the electrical field. For example, if nucleic acid molecules are added in the top chamber in FIG. 2 (between top electrode and the substrate), a current in the direction from the bottom electrode to the top electrode will result in enhanced delivery of the negatively charged molecules into the cells thanks to electrical force.

Although terms such as top, bottom, vertical, horizontal, sideways, upward, or downward are used in describing various aspects of the present invention, it should be understood that such terms are for purposes of more easily describing the invention and do not limit the scope of the invention.

III. Cell Electroporation Configuration and Control

In accordance with embodiments of current based electroporation, preferably biological cells are attached to a substrate that electrical current can pass through. The substrate can be made of an electrical conductive material such as thin film metal, conductive polymer, or conductive glass such as ITO (Indium Tin Oxide). When the substrate is made of an electrical conductive material, it can serve as an electrode as well as the support for cells to attach to.

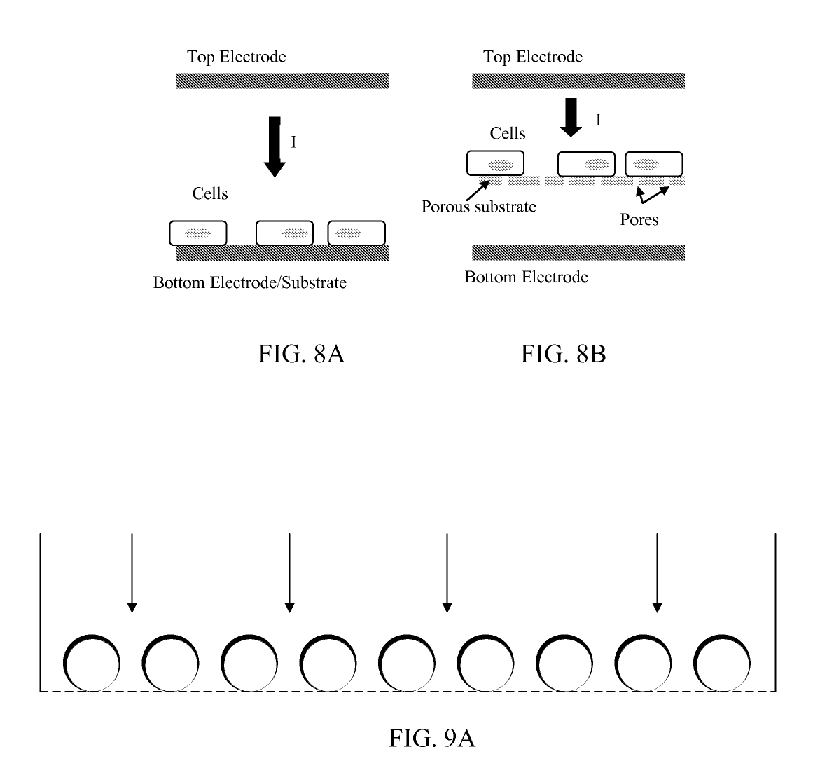

FIG. 8A shows an example of a substrate functioning as an electrode and support for cells in accordance with an embodiment of the invention. A top electrode and a bottom electrode may be provided. The bottom electrode may function as a substrate to support one or more cells. The bottom electrode substrate may be formed of a conductive material, as described elsewhere herein. A current may be provided flowing from the top electrode to the bottom electrode. In alternate embodiments, the current may flow from the bottom electrode to the top electrode. In some instances, in such situations, the cells may be attached to a top electrode.

Alternatively, the substrate can be a thin film of dielectric material (such as polyester, polycarbonate, etc.) with transverse pores on it (i.e., porous membrane). If the substrate is a porous membrane, cells that have at least one pore underneath them are more likely to be electroporated, although cells near a pore can also be electroporated. In some embodiments, the porous membrane may have such a distribution of pores so that a cell on the membrane is likely to cover a pore or be near a pore. For example, the size and/or density of the pores may be great enough to ensure a cell is overlying or near a pore. In some examples, the number of pores provided on a substrate may exceed the number of cells on the substrate.

FIG. 8B shows an example of a porous substrate membrane in accordance with another embodiment of the invention. A top electrode and a bottom electrode may be provided. A porous substrate may be provided between the top and bottom electrodes. Cells may be supported by the porous substrate, and one or more pores may or may not be covered by the cells. In some embodiments, the cells may be provided on top of the porous substrate. A current may flow from the top electrode to the bottom electrode. In alternate embodiments, the current may flow from the bottom electrode to the top electrode. The cells may be attached on top of the porous substrate and/or the bottom of the substrate.

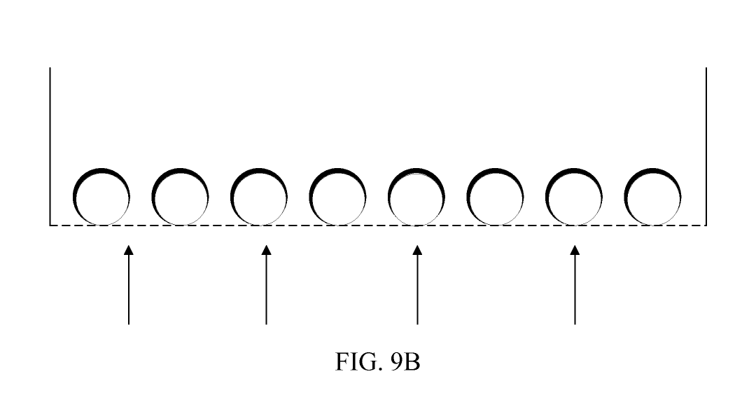

FIG. 9A shows an example of providing a unidirectional current to cells on a substrate in a first direction. The cells may be polarized or somehow directionally arranged so that a first side of the cells has a first set of characteristics, and a second side of the cells has a second set of characteristics. The first and second characteristics may be the same, similar or different from one another. In one example, it may be easier for a foreign substance to be introduced to a host cell through a first side of the cell than the second side of the cell, or vice versa.

In some embodiments, the first side of the cell may be a top side of the cell and the second side of the cell may be a bottom side of the cell. The first side of the cell may be the side of the cell opposite a substrate that the cell is contacting, and the second side of the cell may be the side of the cell contacting the substrate.

In one example, a foreign substance may be driven through a top side of the cell as an electrical current flows from top to bottom. The top side of the cell may have characteristics that may permit the foreign substance to be introduced through the top side. The bottom side may or may not have characteristics that may permit the foreign substance to be introduced through the bottom side.

FIG. 9B shows an example of providing a unidirectional current to cells on a substrate in a second direction. For example, a foreign substance may be driven through a bottom side of the cell as an electrical current flows from bottom to top. This may be preferable in instances when a top side of the cell may have a first set of characteristics that may make it difficult for foreign substances to be introduced into the host cell. In such situations, the bottom side may have a second set of characteristics that are more favorable to introducing a foreign into a substance to the cell.

In some embodiments, a cell electroporation system may allow a unidirectional current to flow from a top electrode to a bottom electrode. In other embodiments, the cell electroporation system may allow a unidirectional current to flow from a bottom electrode to a top electrode. In some instances, the cell electroporation may allow the direction of current to be selected. For example, a user may specify the direction of current flow during a cell electroporation process. In other examples, a direction may be automatically selected by a program based on a transfection condition, such as cell type. The direction of current flow may alternatively be selected based on a measurement taken before or during the cell electroporation process.

The direction of current flow may remain consistent throughout a cell electroporation process. For example, throughout the entire process, the current may always flow from top to bottom or vice versa. Alternatively, the direction may vary during the cell electroporation process. The current direction may be predetermined or may be adjusted in response to an input or measurements. In some instances, the current direction may remain the same for an electrical pulse. Alternatively, the current direction may vary during the pulse.

IV. Device and Apparatus for Inducing Electroporation

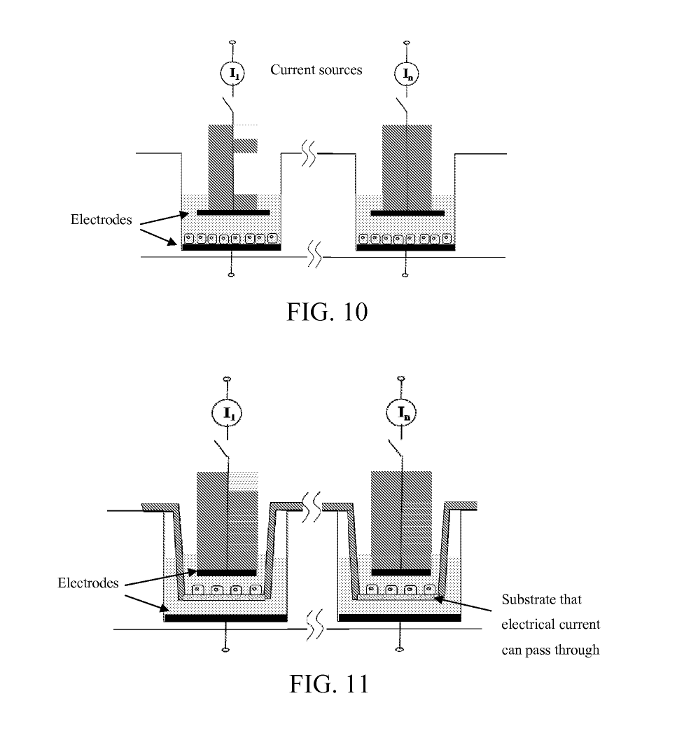

FIG. 10 shows a configuration of a cell electroporation device with cells attached to an electrode in accordance with an embodiment of the invention. As previously discussed, any discussion of cell electroporation and cell transfection may be applied to SACE, and vice versa. For example, a cell electroporation may or may not be used for SACE. In some embodiments, the electroporation device can be in microtiter plate formats, to be discussed in greater detail below. One or more current source may be provided to deliver an electric current to an electrode. In one embodiment, the current source may be in electrical communication with a top electrode. Each current source may be in electrical communication with one or more electrode. The electrodes may or may not be electrically isolated or insulated from one another. In some instances, each electrode may be provided with its own current source. For example if electrodes 1 through n are provided, current sources may be provided for current I₁, . . . Iₙ. The current values for each of the current sources may or may not be the same. In some embodiments, each electrode may have its own current source. Alternatively, each electrode may have one or more multiple current sources. In some instances, at least one current source may be provided to one or more electrode. In some instances, a plurality of current sources may be provided to a plurality of electrodes. Any of the current sources may provide currents with the same or different electrical properties.

A chamber may be provided with a fluid therein, and a population of host cells. The fluid may be an electrically conductive fluid. The chamber may be electrical communication with a first electrode and a second electrode. The first electrode may be in electrical communication with a current source. In some embodiments, the first electrode may be a top electrode. A top electrode may be in contact with the electrically conductive fluid, or any contents of the chamber. The top electrode may or may not contact the host cells. The population of host cells may be contacting the second electrode. In some embodiments, the second electrode may be a bottom electrode.

FIG. 11 shows a configuration of a cell electroporation device with cells attached to a substrate that allows current passage in accordance with another embodiment of the invention. In some embodiments, a chamber may be provided with an electrically conductive fluid therein, and a population of host cells. A current source may be connected to a first electrode contacting the electrically conductive fluid. In some embodiments, the first electrode may be a top electrode.

In some embodiments, the host cells may be contacting a substrate that may enable an electrical current to pass through. The substrate may or may not be a porous membrane. The substrate may be separate from a second electrode in electrical communication with the contents of the chamber. The current may flow from the first electrode to the second electrode. The substrate may be suspended over the second electrode. In some instances, the substrate may be suspended so that it does not or does contact the second electrode. The electrically conductive fluid may or may not flow through the substrate.

In some embodiments, a chamber may include an upper section and a lower section, where the upper section includes a portion of the chamber above the substrate, holding the host cells and with a first electrode disposed therein. The lower section may include a portion of the chamber below the substrate and not holding the host cells, and with a second electrode disposed beneath. In some embodiments, separate lower sections may be provided for each chamber. The areas beneath the substrates for each of the chambers need not be in fluid communication with one another. This may assist with preventing cross-contamination between the chambers. In such situations separate second electrodes may be provided for each chamber.

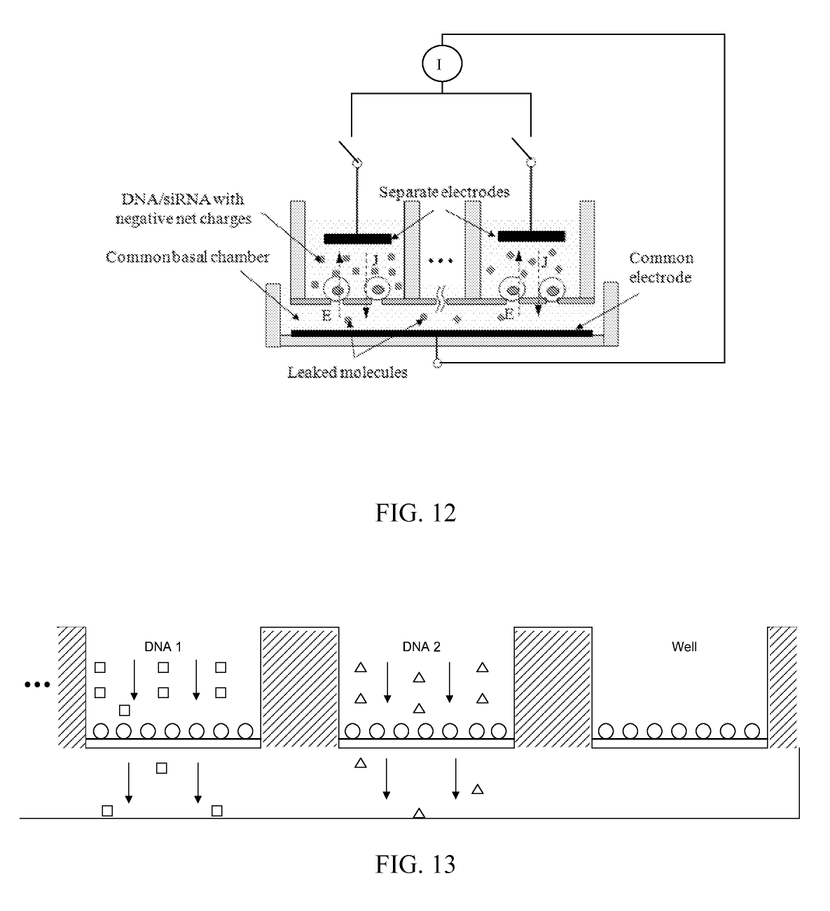

FIG. 12 shows a configuration of a cell electroporation device with a common bottom electrode and a common bottom/basal chamber in accordance with an embodiment of the invention. A plurality of chambers may be provided, wherein each chamber may include a population of host cells and an electrically conductive fluid. A top electrode may be provided to each chamber. In some embodiments, different current sources or the same current source may be provided to each of the top electrodes. A current source may be realized with a current clamping circuit. The population of host cells may rest on a substrate. The substrate may or may not be porous.

A common bottom electrode and basal/bottom chamber may be provided. An electrically conductive fluid may be provided within the common basal chamber. The electrically conductive fluid may or may not pass through the substrate. The chambers may or may not be in electrical communication with the common basal chamber. A foreign substance may be provided for introduction into the host cells. In some embodiments, the foreign substances may have a net charge. For example, they may have negative or positive net charges. In one example, the foreign substances may be DNA or siRNA, which may have negative net charges. Other examples of foreign substances include, but are not limited to, microRNA, peptides, proteins, small molecules, and nanoparticles.

The common bottom electrode and the top electrodes may be part of a current-clamping circuit. An electrical field E may be generated, which may result in electrodiffusive movement J. For foreign substances that have a negative net charge (e.g., nucleic acid molecules), E and J may be in opposite directions. For example, if an electrical field E is directed upwards, the electrodiffusive movement J may be directed downward, which may drive the foreign substances downwards to the host cells. Alternatively, if the electrical field were directed downwards, the electrodiffusive movement may be directed upwards, which may be used to drive foreign substances beneath the host cells into the cells. In some instances, when the electrodiffusive movement is downward, some of the foreign substances may be driven past the cells into the basal chamber. In some instances, if the foreign substances have a positive net charge, the electrodiffusive movement may be in the same direction as the electric field.

Since the transfection wells may share a common electrode and a common basal medium chamber, it is possible that certain amount of molecules loaded in individual wells can leak into the common chamber. This could complicate a transfection experiment since cells in a particular unit can be transfected with molecules from the other wells through the common chamber. This leakage issue can be minimized by growing cells into a fully confluent monolayer. In some embodiments, a population of host cells may be provided within a unit so they form a fully confluent monolayer. In other embodiments, the host cells may form more than a monolayer. In alternate embodiments, the cells need not be fully confluent. The host cells may cover about 10% or more, 25% or more, 50% or more 75% or more, 80% or more, 85% or more, 90% or more, 95% or more, 97% or more, 99% or more, 99.5% or more, or 100% of a bottom of the unit. In some instances, the host cells may be evenly distributed on a surface of the unit. For example, the host cells may be evenly distributed on the bottom surface of the unit. Alternatively, the host cells may have a varying distribution on the surface.

In some embodiments, a cell density of 500 to 1,000,000 cells per square centimeter may be provided. In some other embodiments, a cell density may fall between 1,000 to 500,000 cells per square centimeter, or 5,000 to 100,000 cells per square centimeter. In some instances, a cell density of 100 or greater, 200 or greater, 500 or greater, 1,000 or greater, 5,000 or greater, 10,000 or greater, 25,000 or greater, 50,000 or greater, 75,000 or greater, 100,000 or greater, 150,000 or greater, 200,000 or greater, 300,000 or greater, 500,000 or greater, or 1,000,000 cells per square centimeter may be provided. A cell density may be selected to provide a desired cell confluence. In some instances, the cell density may depend on the host cell type and/or size.

Furthermore, the current electroporation can minimize the leakage effect as well. As indicated before, in a current electroporation mediated transfection, nucleic acids such as DNAs and siRNAs are introduced to cells primarily through electrodiffusive force. As indicated in FIG. 12, one electrical field E, or electrical current I, is in direction from the common bottom electrode to a top electrode, the electrodiffusive force on the negatively charged nucleic acid molecules, which typically are loaded in individual wells, facilitates delivery of the molecules from the top chambers into the cells, and in the same time, prevent molecules leaked into the bottom common chamber from entering the cells, as shown in FIG. 13. This effect can substantially reduce potential cross-talk due to the use of a common chamber. On the other hand, nucleic acid molecules can be loaded into the common chamber, and direction of applied electrical current/field can be adjusted such that it drives the molecules into the cells from the common bottom chamber.

The cell electroporation method works better when electrical current can transverse cells. When a porous membrane made of dielectric material is used, cells with at least one micropore underneath them are more likely to be electroporated. Similarly, cells that are in the vicinity of a pore, even if the pores are not directly beneath them have an increased chance of being electroporated. Cells without pores underneath them and that are not in the vicinity of micropores which allows passage of electrical currents, are less likely to be affected and electroporated. As such, in order to increase or maximize transfection efficiency, procedures can be taken to ensure most cells in a well will sit on top of one or more pores, or sit close to one or more pores.

FIG. 13 shows an example of a unidirectional current used within a cell electroporation device that may assist with preventing cross-contamination. A plurality of chambers may be provided. The chambers may be formed of wells on a microtiter plate. A population of host cells may be provided in a plurality of chambers. The population of host cells may be supported by a substrate. In some embodiments, the substrate may be a porous membrane. The plurality of chambers may also contain an electrically conductive fluid. In some instances, the electrically conductive fluid may flow through the porous membrane.

In some embodiments, a foreign substance may be introduced to a chamber of an electroporation device. The foreign substance may be the same, or may be differ from chamber to chamber. Any number of chambers of an electroporation device may have the same or differing foreign substances. In one example, a first foreign substance, e.g., DNA1 may be introduced to a first chamber and a second foreign substance, e.g., DNA2 may be introduced to a second chamber. The foreign substances may be driven to the host cells of their respective chambers. In some instances, some of the foreign substances may pass through the substrate. For example, DNA1 may be driven through the substrate of the first chamber into a common chamber below, and DNA2 may be driven through the substrate of the second chamber into the common chamber. A downward unidirectional current may advantageously drive DNA1 and DNA2 downward to the bottom of the common chamber. This may prevent or reduce the amount of the foreign substances from traveling sideways and/or upwards and contaminating the other adjacent chambers. By driving foreign substances downwards toward the bottom of the common chamber, cross-contamination may be reduced or prevented.

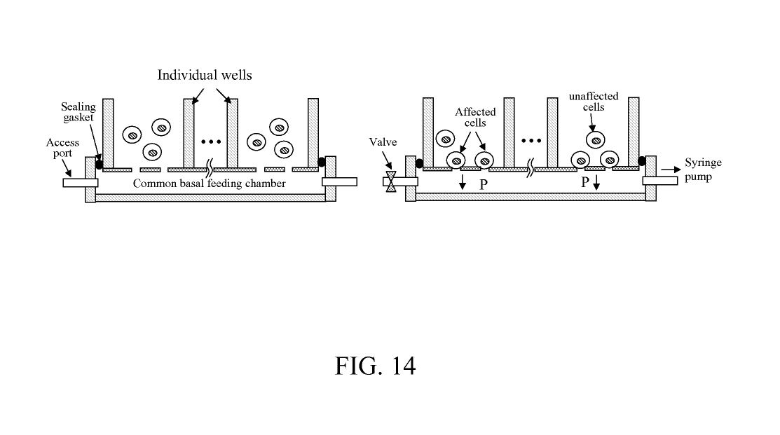

FIG. 14 illustrate a method that use pressure to position cells on one or more pores, as previously discussed. In a typical procedure, a pressure difference (typically 0.1-1 par) may be generated across the porous membranes; cells in suspension are then added to individual wells; the cells will may then be pulled toward micropores; then the pressure may be withdrawn, the cells are allowed to sit on the pores for an certain period of time until they attach to the substrate. After this, current electroporation can be performed. Using this method, cells can be selectively electroporated and thus transfected, since these cells not in the vicinity of a pore are less likely to be affected by the electrical current. One example of using selective transfection of the same type of cells, or different type of cells, is to transfect certain molecules into a portion of the cells, then study interaction between the transfected cells and the unaffected cells.

In some embodiments, the pressure differential may be provided by a negative pressure source in communication with the space below the porous membrane. Alternatively, a positive pressure source may be provided in communication with the space above the porous membrane. An example of a positive pressure source may be a pump. In some instances, a combination of both negative and positive pressure sources may be utilized.

FIG. 14 provides an illustration of using pressure to position cells on specific locations of a porous membrane. In one example, a plurality of wells may be provided, wherein the wells may contain a population of host cells and a fluid. In some embodiments, the host cells may be provided in suspension. The plurality of wells may feed into a common basal feeding chamber. A sealing gasket may be provided, which may effectively seal the common basal feeding chamber. An access port may be provided to the common basal feeding chamber.

A valve may be provided as well as a syringe pump, which may effectively lower the pressure within the common basal feeding chamber. Any other pumping mechanism or means of lowering pressure within the common basal feeding chamber may be employed. The common basal feeding chamber may be in communication with a vacuum or any other negative pressure source that may reduce the pressure within the basal feeding chamber.

A plurality of wells may be disposed over the common basal feeding chamber. The wells may be in fluid communication with the common basal feeding chamber. The interface between the wells and the common basal feeding chamber may include a porous substrate, or other surface with holes or pores. A population of host cells may be provided within the wells. In some embodiments, a fluid may be provided in the wells and in the common basal feeding chamber. The fluid may flow through the pores or holes between the wells and common basal feeding chamber. When a negative pressure is provided to the common basal chamber, the host cells may be drawn toward the pores or openings. The pores or openings may have a desired configuration or distribution. Affected cells may be adhered to the porous substrate. Unaffected cells may remain in suspension within the wells. In some embodiments, cells may be unaffected when they are provided in excess of the pores on the substrate. The pressure may be used to position cells on specific locations of the porous substrate.

A cell electroporation device can have any configuration that may receive or contain a population of host cells. Any aspects, characteristics, features, or steps known in the art may be utilized in the cell electroporation device. See, e.g., U.S. Pat. No. 7,687,267 which is hereby incorporated by reference in its entirety. In some embodiments, a cell electroporation device may be configured with a plurality of chambers that may be configured to receive or contain the host cells. A chamber may have any shape that may contain or confine the host cells. For example, the chamber may be in the form of a well. The chamber may have an open top, or may have a closed top. In some embodiments, the chamber may have a top that may include an opening that may be smaller than or equal to the cross sectional area of the chamber. The chamber may have an open bottom or a closed bottom. In some embodiments, the chambers may all be open or have an opening along the same side (e.g., top, side, or bottom). Alternatively, the orientation of an opening of a chamber may vary. The chambers and/or any portion of the electroporation device may be formed of any material that may allow a population of host cells to be contained and/or confined within the electroporation device. A solid support may be provided. The solid support may include plastic polymer, glass, cellulose, nitrocellulouse, semi-conducting material, electrically and/or thermally insulating material, metal, or any combination thereof.

The chamber may have any cross-sectional shape. For example, the chamber may have a circular shape when viewed from above, an elliptical shape, a triangular shape, a square shape, a rectangular shape, a pentagonal shape, a hexagonal shape, an octagonal shape, a crescent shape, or any other polygonal or non-polygonal shape. A bottom of the chamber may be flat, tapered, rounded, conical, curved, bent, or have any other. The bottom of the chamber may be solid and/or fluid impermeable. Alternatively, it may be porous or include one or more pores, openings, channels, holes, paths, or be formed of a permeable material that may enable a fluid to travel through the bottom. Any description of the porous membrane may be applied to any substrate that may include a pathway enabling a fluid to enter and/or pass through the substrate. Example sof porous membranes include, but are not limited to, polycarbonate (PC), Polyethylene terephthalate (PET), and polytetrafluoroethylene (PTFE) membranes.

A chamber may be of any size. For example, the chamber may be the size of a standard well for a microtiter plate. In some embodiments, a chamber may have a volume of about 100 nL, 1 μL, 10 μL, 100 μL, 200 μL, 300 μL, 500 μL, 750 μL, 1 mL, 1.5 mL, 2 mL, 3 mL, 4 mL, 5 mL, 7 mL, or 10 mL. For example, the chamber volume may fall within 10 mL to 10 mL. In some embodiments, the depth of the chamber may fall within 1 μm to 5 cm. For example, the depth of the chamber may be about 10 μm, 100 μm, 250 μm, 500 μm, 750 μm, 1 mm, 2 mm, 3 mm, 5 mm, 7 mm, 1 cm, 1.2 cm, 1.5 cm, 2 cm, 3 cm, 4 cm, or 5 cm. In some embodiments, the diameter, width, and/or length of the chamber may fall within 1 μm to 5 cm. For example, the diameter, width, and/or length of the chamber may be about 10 μm, 100 μm, 250 μm, 500 μm, 750 μm, 1 mm, 2 mm, 3 mm, 5 mm, 7 mm, 1 cm, 1.2 cm, 1.5 cm, 2 cm, 3 cm, 4 cm, or 5 cm.

The chambers may be spaced apart from one another so that they are relatively close packed or spread apart. In some embodiments, the chambers may have a distance of about 10 μm, 100 μm, 250 μm, 500 μm, 750 μm, 1 mm, 2 mm, 3 mm, 5 mm, 7 mm, 1 cm, 1.2 cm, 1.5 cm, 2 cm, 3 cm, 4 cm, or 5 cm from center to center. In some embodiments, the distance between the chambers from center to center may be greater than the diameter, length, or width of the chamber. The chambers may or may not be evenly spread apart. In some embodiments, groups of chambers may be evenly spaced apart.

In some embodiments, all of the chambers of a cell electroporation device may have the same size or shape. Alternatively, the size and/or shape of the chambers may vary for a given electroporation device. In some instances, groups of chambers may be provided that may have the same size and/or shape, which may or may not be different from the size and/or shape of the chambers of other groups.

Any number of chambers may be provided. For example, one, two, three, four, or more chambers may be provided. In some instances, the number of chambers may correspond to a number of a traditional microtiter plate, such as 6 chambers, 12 chambers, 24 chambers, 48 chambers, 96 chambers, 384 chambers, 1536 chambers, 3456 chambers, or 9600 chambers. Some cell electroporation devices may have about 2, 4, 9, 15, 20, 25, 30, 36, 50, 75, 100, 150, 200, 250, 300, 350, 400, 500, 600, 700, 800, 900, 1000, 1200, 1500, 2000, 3000, or more chambers.

The chambers may be provided in any configuration in relation to one another. For example, the chambers may be arranged in one or more rows, or one or more columns. In some embodiments, the chambers may be provided within an array, with one or more rows and one or more columns. In some embodiments, an x-axis and a y-axis may be defined for an electroporation device such that the x and y axes are orthogonal to one another when viewing the device from above. One or more rows may be provided parallel to the x-axis and one or more columns may be provided parallel to the y-axis. In some embodiments, a plurality of rows, or a plurality of columns may be provided in a staggered manner. This may enable the chamber to be more closely packed. Alternatively, they may be provided so that each chamber within a row forms a column with a chamber in adjacent row. The chambers may be distributed in any other manner. For example, they may be provided randomly, in a concentric pattern (e.g., concentric circles, squares, triangles, or any other shapes), forming bent or wavy lines or rows, or forming spoke patterns.

The simple configurations described above can be implemented in various formats. In one embodiment, the electroporation device may be a plate, such as 6-well, 12-well, 24-well, 48-well, 96-well, 384-well, 1536-well, 3456-well, or 9600-well microtiter plate devices, for electroporating and subsequently transfection of various molecules into adherent biological cells, regardless whether the cells are in proliferating, post-confluent or fully differentiated states.

Various configurations of cell electroporation devices from processing cells in microtiter plate formats, such as 6/12/24/48/96/384/1536/3456/9600-wells could be utilized incorporating features and configurations for chambers and electroporation devices as previously described. In some embodiments, the device configuration shown in FIG. 12 may be particularly useful in high-throughput transfection, since such a configuration may make it possible to implement the concept in 96-well or higher throughput micro plate formats.

In some embodiments, the overall plate dimensions may be about 127.8 mm long by 85.6 mm wide. In some embodiments, the plate dimensions may match the standard dimensions for a microtiter plate. In other embodiments, the plate dimensions may vary. For example, one or more of the plate dimensions may be about 10 mm, 20 mm, 30 mm, 50 mm, 70 mm, 80 mm, 100 mm, 110 mm, 120 mm, 130 mm, 150 mm, 175 mm, 200 mm, 250 mm, 300 mm, or 500 mm. An overall plate may be rectangular, square, circular, triangular, hexagonal, octagonal, or have any other regular or irregular shape. In some instances, the plate thickness may be about 1 mm or less, 2 mm or less, 3 mm or less 5 mm or less, 7 mm or less, 10 mm or less, 1.2 cm or less, 1.5 cm or less, 2 cm or less, 3 cm or less, or 5 cm or less.

The accompanying drawings illustrate various designs of devices and apparatus for using electrical current to induce electroporation in cells in various plate format. Any of the various designs and apparatuses may incorporate characteristics, features, or components of any of the embodiments described in U.S. Pat. No. 7,687,267, which is hereby incorporated by reference in its entirety.

Any types of cells may be provided within a cell electroporation device or plate. As previously discussed, the cells may be primary cells, engineered cells, and/or cell lines. For example, cells that are representative of a distinct organism to be tested may be provided. Any cells that are isolated from the test organism, whether they are cultured in vitro as primary culture or cell lines, or isolated from different tissues of that organism, can be provided in a cell electroporation device. Such cells may share a common characteristic, and hence may be considered to be the same type. In some instances, chambers of a cell electroporation device may contain cells derived from a single tissue that is under investigation.

In some embodiments, the cells provided in a cell electroporation device and/or chamber of an electroporation device may be substantially homogenous. Alternatively, the cells may be heterogeneous, within a single chamber, or over the multiple chambers of the electroporation device. The cells can be living or dead cells; eukaryotic or prokaryotic cells; embryonic or adult cells; or cells of ectodermal, endodermal or mesodermal origin. The cells loaded in the tube can also be freshly isolated cells, cultured cells in either primary or secondary cultures, or cells of an established cell line. Furthermore, the cells may be wildtype, genetically altered or chemically treated cells.

Cells contained in a cell electroporation device, or of a subset of chambers within a cell electroporation device, may differ in one or more of the characteristics selected from a group comprising genotypic characteristics, species origin, developmental stage, developmental origin, tissue origin, cell-cycle point, chemical treatment and disease state. Whereas the species origin may be selected from the group consisting of human, mouse, rat, fruit fly, worm, yeast and bacterium, suitable tissues from which cells are derived are blood, muscle, nerve, brain, heart, lung, liver, pancreas, spleen, thymus, esophagus, stomach, intestine, kidney, testis, ovary, hair, skin, bone, breast, uterus, bladder, spinal cord, or various kinds of body fluids. The cells contained in the chambers of the cell transfection device may also differ in developmental stage including embryo and adult stages, as well as developmental origin such as ecotodermal, mesodermal, and ectodermal origin. As such, the cells provided in the cell transfection device may encompass embryonic cells, adult cells, primary cells, cell lines, tissue cells, mammalian cells, zoo cells, personal cells, genetically altered cells, chemically treated cells, and disease cells. Preferred disease cells may include cancer cells.

In some embodiments, the cells provided may correspond to a distinct biological organism. Exemplary organisms include members of the plant or animal kingdom, and microorganisms such as viruses, bacteria, protozoa, and yeast. The cells may be provided from a unicellular or a multi-cellular organism. The cells of a human being may be included. Cells of a model organism including but not limited to mouse, rat, fruit fly, worm, yeast, bacteria, corn and rice may be included.

Cells derived from a distinct mammal may be provided for cell transfection. Non-limiting examples of mammals are primates (e.g. chimpanzees and humans), cetaceans (e.g. whales and dolphins), chiropterans (e.g. bats), perrisodactyls (e.g. horses and rhinoceroses), rodents (e.g. rats), and certain kinds of insectivores such as shrews, moles and hedgehogs. In some instances, the cell transfection device may contain human cells of various types.

In some embodiments, the cell population may be representative of a specific body tissue from a subject. The types of body tissues include but are not limited to blood, muscle, nerve, brain, heart, lung, liver, pancreas, spleen, thymus, esophagus, stomach, intestine, kidney, testis, ovary, hair, skin, bone, breast, uterus, bladder, spinal cord and various kinds of body fluids. Non-limiting exemplary body fluids may include urine, blood, spinal fluid, sinovial fluid, ammoniac fluid, cerebrospinal fluid (CSF), semen, and saliva.

Also embodied in the subject invention are cells corresponding to different developmental stages (embryonic or adult) of an organism, or more specifically corresponding to various developmental origins including ectoderm, endoderm and mesoderm.

Further provided by the present invention are freshly isolated cells, cells derived from a plurality of primary cultures (i.e. “primary cell array”) or subcultures generated by expansion and/or cloning of primary culture (i.e. “cell line array”). Any cells capable of growth in culture can be used. Non-limiting examples of specific cell types include connective tissue elements such as fibroblast, cells of skeletal tissue (bone and cartilage), cells of epithelial tissues (e.g. liver, lung, breast, skin, bladder and kidney), cardiac and smooth muscle cells, neural cells (glia and neurones), endocrine cells (adrenal, pituitary, pancreatic islet cells), melanocytes, and many different types of haemopoietic cells. Of particular interest is the type of cell that differentially expresses (over-expresses or under-expresses) a disease-causing gene. As is apparent to one skilled in the art, various cell lines may be obtained from public or private repositories. The largest depository agent is American Type Culture Collection (http://www.atcc.org), which offers a diverse collection of well-characterized cell lines derived from a vast number of organisms and tissue samples.

Other types of cells may be derived from individuals of a family, or individuals from different generations within the same pedigree. Cell arrays of this category may be especially useful for forensic and parental identification.

Cells associated with a particular disease or with a specific disease stage may be provided for cell transfection. The association with a particular disease or disease stage may be established by the cell's aberrant behavior in one or more biological processes such as cell cycle regulation, cell differentiation, apoptosis, chemotaxsis, cell motility and cytoskeletal rearrangement. A disease cell may also be confirmed by the presence of a pathogen causing the disease of concern (e.g. HIV for AIDS and HBV for hepatitis B). The types of diseases involving abnormal functioning of specific types of cells may include but are not limited to autoimmune diseases, cancer, obesity, hypertension, diabetes, neuronal and/or muscular degenerative diseases, cardiac diseases, endocrine disorders, and any combinations thereof.

Other categories of cells provided for electroporation may include “genetically altered” or “chemically treated” cells. A cell is “genetically altered” as compared to a wildtype cell when a genetic element has been exogenously introduced into the cell other than by mitosis or meiosis. The element may be heterologous to the cell, or it may be an additional copy or improved version of an element already present in the cell. Genetic alteration may be effected, for example, by transfecting a cell with a recombinant plasmid, or other polynucleotide delivery vehicle through any process known in the art, such as electroporation, viral infection, calcium phosphate precipitation, or contacting with a polynucleotide-liposome complex. When referring to genetically altered cells, the term refers both to the originally altered cell, and to the progeny thereof. A preferred altered cell is one that carries a reporter gene to effect drug screening, cellular pathway delineation, and/or antibody selection.

Chemically treated cells may be treated with distinct chemical agents or a particular combination of chemical agents. As used herein, a “chemical agent” is intended to include, but not be limited to a biological or chemical compound such as a simple or complex organic or inorganic molecule, a peptide, a protein (e.g. antibody), a polynucleotide (e.g. antisense oligonucleotide), a ribozyme and its derivative. A vast array of compounds can be synthesized, for example polymers, such as polypeptides and polynucleotides, and synthetic organic compounds based on various core structures, and these are also included in the term “chemical agent”. In addition, various natural sources can provide compounds for screening, such as plant or animal extracts, and the like.

Some examples of cell lines may include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRCS, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-MeI 1, 2, 3 . . . 48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR. Cell lines are available from a variety of sources known to those with skill in the art (see, e.g., the American Type Culture Collection (ATCC) (Manassus, Va.)).

As previously discussed, the same category of cells or types of cells may be provided throughout the transfection device. Alternatively, the same category of cells or types of cells may be provided for groups of chambers within the cell transfection device, or within individual chambers of the cell transfection device. In some embodiments, multiple categories or types of host cells may be provided across a cell transfection device, within groups of chambers of a cell transfection device, or within individual chambers of a cell transfection device.

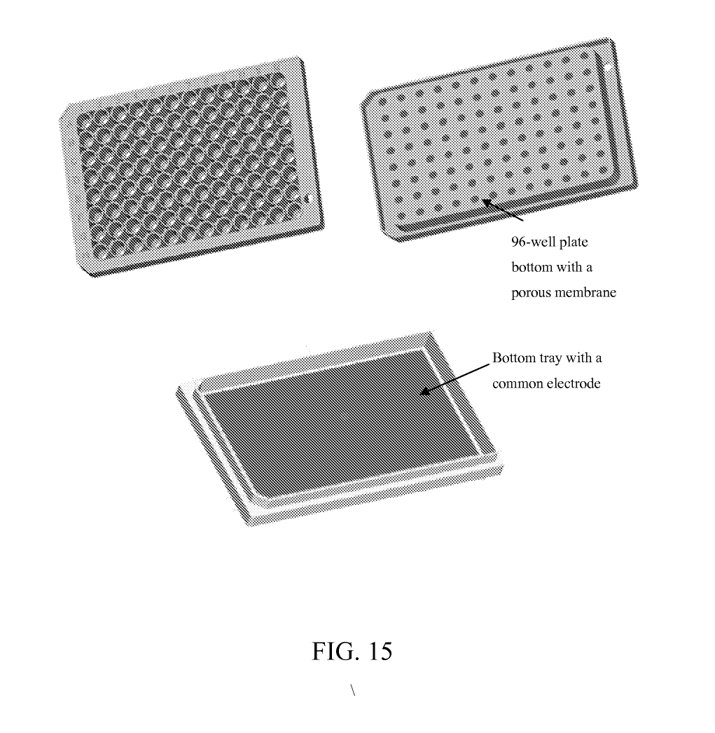

FIG. 15 shows an example of a cell electroporation device in a microtiter plate format. In one embodiment, a well plate and a bottom tray may be provided. The well plate may have any of the configurations described, such as a 96 well plate. Any description of a 96 well plate may apply to the other plates. A top view of the well plate and a bottom view of the well plate is provided. The openings at a top of a well plate may have a greater cross sectional area than the opening at the bottom of a well plate. In some embodiments, a porous membrane may be provided at the bottom openings of the well plate.

The well plate may have a lip or supports that may protrude from the sides of the well plate. The supports may be provided so that the well plate may be suspended within the bottom tray. The support may allow the well plate to be suspended over the bottom of the tray so as to not contact the bottom electrode. Alternatively, the well plate may rest within the bottom tray and contact the bottom electrode. In other embodiments, the well plate may have protrusions or features on the bottom of the well plate that may allow most of the bottom of the well tray to remain suspended over the bottom electrode.

The bottom tray may include a common electrode. The common electrode may cover the bottom surface of the bottom tray. In some embodiments, the common electrode may be formed of a simply connected continuous piece, while in other embodiments, the electrode may be formed of discontinuous pieces or may have holes therein. The dimensions of the bottom tray allow the well plate to fit within the bottom tray. The bottom tray may include walls that may be high enough to contain a fluid therein. In some instances, the walls may be high enough to allow the well plate to be suspended without contacting the bottom electrode. The walls may interfere with the well plate supports so that the well plate may or may not shift sideways with respect to the bottom tray. A well plate may fit into a bottom tray so that it does or does not form an air tight connection within the bottom tray.

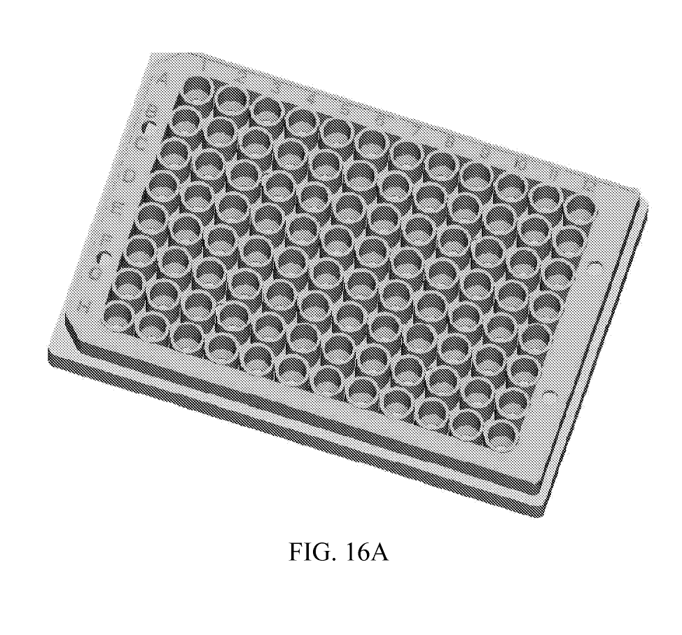

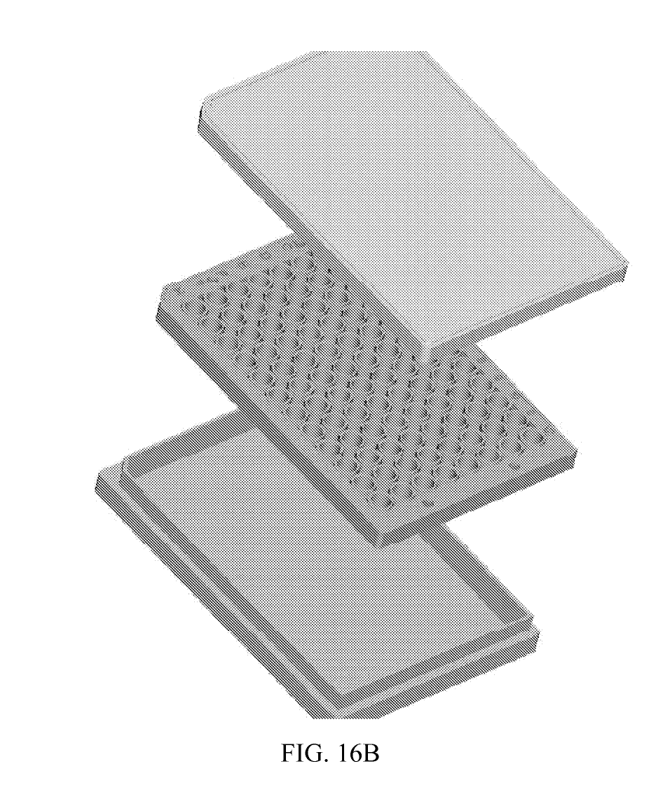

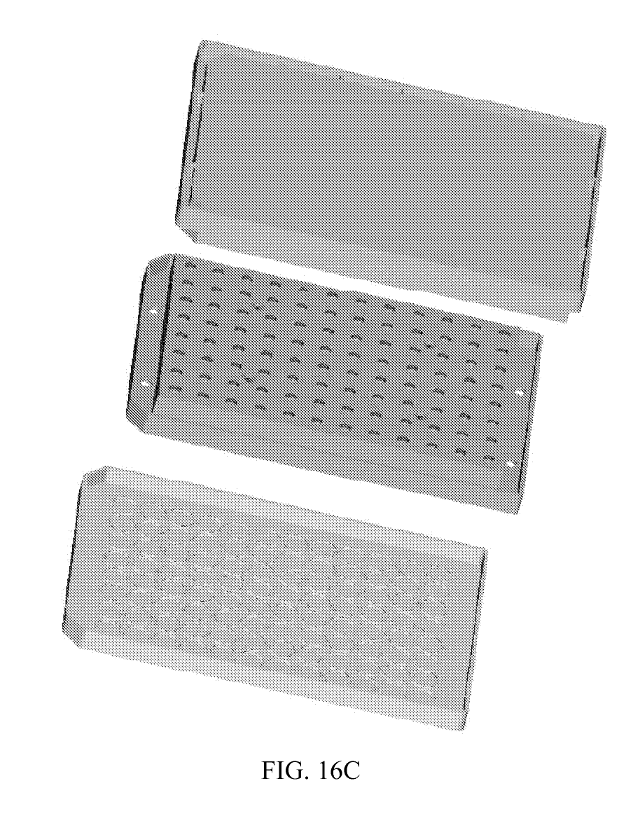

FIGS. 16A-C show views of an embodiment of the cell electroporation device. FIG. 16A shows a top view of a 96 well plate. The wells may have circular configurations and be provided in a 8×12 array. In some embodiments, the spaces between the wells may be hollow or may be solid. In some instances, the spaces between the wells may include the same material as forming the rest of the well plate. Alternatively, a different material may be used, which may assist with thermal insulation, conduction, or cooling. In some instances, air spaces may be provided between wells. Preferably, the wells may be formed of a material that may electrically isolate or insulate the contents of the wells from one another. In some embodiments, the material between the wells may allow such electrical isolation or insulation.

A well plate may optionally include one or more label. The label may indicate a row and/or column of the well plate. For example, a row may be indicated by letters (e.g., A-H) and a column may be indicated by numbers (e.g., 1-12). Each well may be addressable by the label. In some embodiments, each well may have a unique label. Alternatively a plurality of wells, or groups of wells may have a unique label. In some embodiments, the labels may correspond to a visual representation of the well plate, described elsewhere herein.

FIG. 16B shows a plurality of components of a cell electroporation device. For example, a top electrode, well plate, and bottom tray may be provided. A top electrode assembly may fit over the well plate, which may fit into the bottom tray. In some embodiments, the top electrode may cover the well plate. In some instances, a plurality of top electrodes may be provided in electrical communication with the well plate. For example, each well may have its own electrode. Alternatively, one electrode may be provided for a plurality of wells or a group of wells. A single top electrode may be provided for all of the wells in the well plate.

FIG. 16C shows another example of a plurality of components that may be provided for a cell electroporation device. A bottom view is provided of a bottom tray, well plate, and top cover. In some embodiments, the bottom of the bottom tray may be flat. The well plate may fit into the bottom tray such that a portion of the wells are within the bottom tray. A top cover may cover the well plate. The top cover may include top electrodes that may be electrical communication with the contents of the well plate.

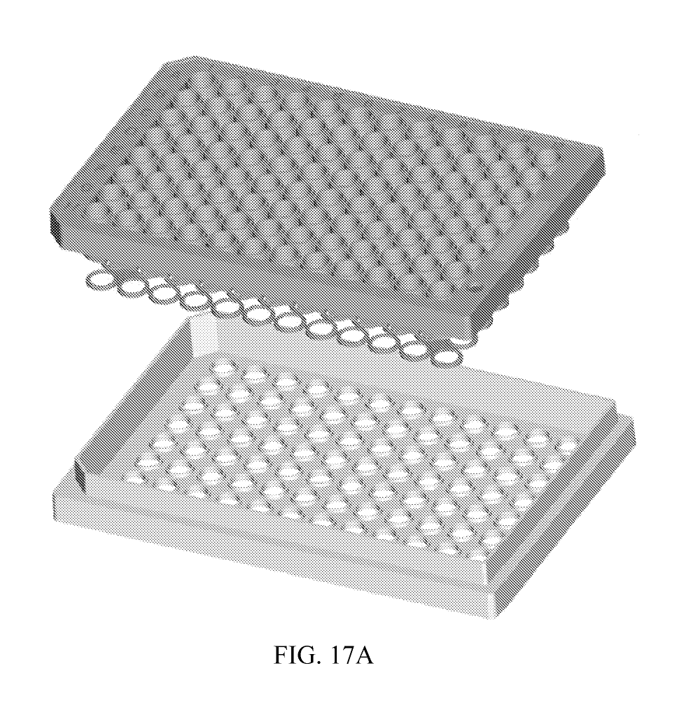

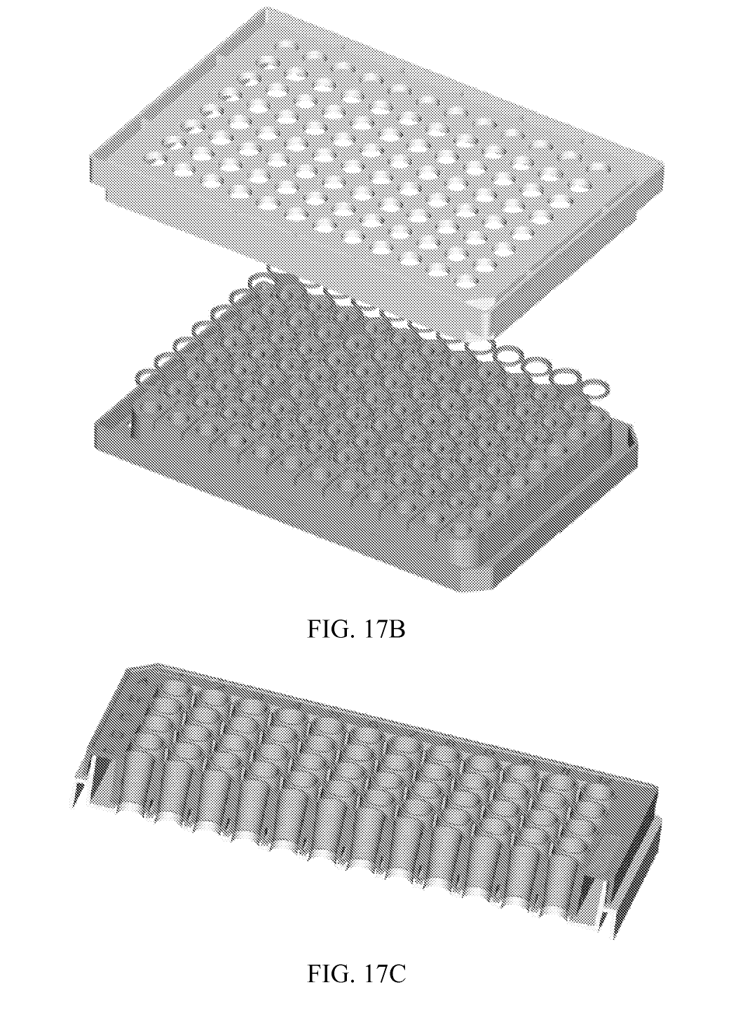

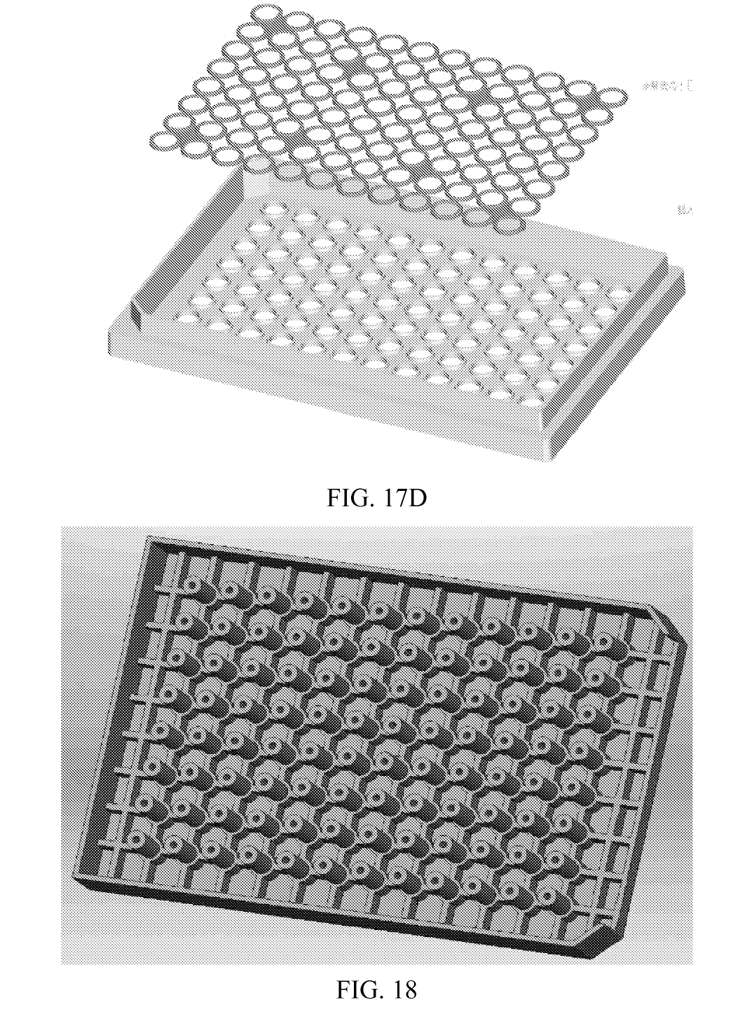

FIGS. 17A-D show views of another embodiment of the cell electroporation device. FIG. 17A shows a top view of a well plate, a seal/gasket, and a bottom tray with individual wells. In some embodiments, the seal may be positioned between the top well plate and the bottom tray. This may allow the two components to be connected and form a through passage. In some embodiments, the seal/gasket separates wells from each other in the bottom chamber, which can be used to avoid cross contamination when transfecting different foreign substance into different wells from the bottom chamber and/or when performing one or more dowstream assays. In some embodiments, the wells in the bottom tray are open, which make it possible to be used as bottom electrode when the electrode is bonded at the bottom, or to be used as receiver tray when a solid plastic film is bonded.

FIG. 17B shows a bottom view of the bottom tray, the seal, and the top tray. The seal may be formed with an opening for each well, and may be aligned with the bottom opening of the top tray, and an opening of the bottom tray. FIG. 17C shows a cut-away cross section of the cell electroporation device where the well plate is fitted into the bottom tray with the seal provided therebetween. The seal may be provided so that the interior cross-sectional area of the well plate and the bottom tray may align with one another. The bottom tray and the top well plate may have interlocking portions that may enable the top well plate to rest within the bottom tray.

FIG. 17D shows an additional view of the seal/gasket and the bottom tray. In some embodiments, the seal may include a plurality of openings that may correspond to the openings of the bottom tray. The size and/or position of the openings of the seal may correspond to the openings in the tray. In some embodiments, the seal may be formed of ringlike shapes that may be provided as an array. In some embodiments, gaps may be provided between the rings. Alternatively, the areas between the rings may be filled. In some embodiments, some of the areas between the rings may be filled while others are open. This may provide some stability in the shape while using fewer materials.

In accordance with an aspect of the invention, a cell transfection apparatus may be provided. The cell transfection apparatus may or may not be a SACE apparatus. The cell transfection apparatus may comprise (a) a plate having a plurality of chambers, each configured to receive and confine a population of host cells; (b) a plurality of electrodes, each of which being configured to be in electrical communication with a corresponding chamber of said plate; (c) an input unit adapted to receive input of one or more transfection parameters from a user; and (d) a controller for processing said input and effecting an electrical current for a defined period of time to at least one electrode of said plurality, based on the input of said one or more transfection parameters from said user.

A transfection parameter may be a factor or characteristic of a transfection. A transfection parameter may affect the transfection that may occur in accordance with an embodiment of the invention. A transfection parameter may have a default value, may be automatically determined, or may be manually inputted. In some embodiments, a transfection parameter may have a default value that may automatically or manually be altered to effect a particular transfection. A transfection parameter may or may not include an electrical property (e.g, membrane resistance, voltage, duration of current flow, rate of current flow, amplitude of current flow, number of electric pulses, shape of electrical pulses). A transfect parameter may or may not include other properties (e.g., cell type, choice of transfection plate). One or more transfection parameters is selected from the group consisting of cell type, membrane resistance, voltage, duration of current flow, rate of current flow, amplitude of current flow, number of electric pulses, shape of electrical pulses, and choice of transfection plate. In some embodiments, the choice of transfection plate may be selected from the group consisting of dimensions of transfection plate, transfection plate materials, cell attachment substratum, choice of porous membrane, and size of chamber. The input unit of the apparatus may comprise a member selected from the group consisting of a touch screen, a keyboard, a button, a switch, a mouse, a trackball, a stick, or a remote controller. A user may select an option (e.g., protocol) that may have predefined transfection parameters. The user may or may not alter one or more transfection parameter. Alternatively, a user may be able to create a protocol comprising one or more transfection parameter.

The cell apparatus may include two way communication that is provided between the input unit and controller. The input unit communicates with the controller via a wired connection or via wireless communications. The input unit and the controller may communicate directly with one another or through a network such as a local area network or a wide area network. The input unit may be provided as an integral part of an assembly comprising the controller. Alternatively, the input unit may be provided external to an assembly comprising the controller.

In some embodiments, the controller comprises one or more processor. An electrode may be positioned beneath the plate. The plurality of chambers may be arranged in an array comprising one or more row and one or more column.

Another aspect of the invention may provide a method for transfecting a population of host cells. The method may include the steps of providing a cell transfection apparatus as described elsewhere herein, (b) receiving input of one or more transfection parameters from a user, and (c) processing said input and effecting an electrical current for a defined period of time to said population of host cells, based on the input of said one or more transfection parameters from said user. The method may also include the step of providing a foreign substance to the cell transfection apparatus. The foreign substance may be selected from the group consisting of DNA, RNA, siRNA, microRNA, peptide, protein, small molecules, or nanoparticles.

A method for transfecting a population of host cells may be provided in accordance with another embodiment of the invention. The method may comprise the steps of providing a cell transfection apparatus as described elsewhere herein, wherein the apparatus comprises a touch screen having a visual representation of the plate having a plurality of chambers; receiving a user input through the touch screen, wherein the input defines a transfection parameter relating to at least one chamber of said plate; and processing said input and effecting an electrical current for a defined period of time to said chamber, based on the input of said transfection parameter from said user.

FIG. 18 shows an example of an electrode configuration in accordance with an embodiment of the invention. The electrode configuration may be a top electrode. A plurality of electrodes may be provided. The electrodes may protrude from a common base. In some embodiments, the electrodes may be mounted on protrusions from the common base. The electrodes may form a cylindrical shape or a disc shape. In some embodiments, each electrode may be sized or shaped to fit within a well of a well plate. For example, electrodes may have a circular cross sectional shape, although any of the other shapes discussed elsewhere herein may be provided. The size of the electrodes may be equal to or smaller than the interior of the wells. For example, one electrode may be provided for one well. Alternatively, a plurality of electrodes may be provided for one well, one electrode may be provided for a plurality of wells, or a plurality of electrodes may be provided for a plurality of wells.

In one example, 96 electrodes may be provided in an array, corresponding to wells within a 96-well plate. If N wells are provided on a plate, N electrodes may be provided. Alternatively, any other number of electrodes may be provided. The electrodes may be provided on a base. The base may be shaped or fitted to go over the well plate.

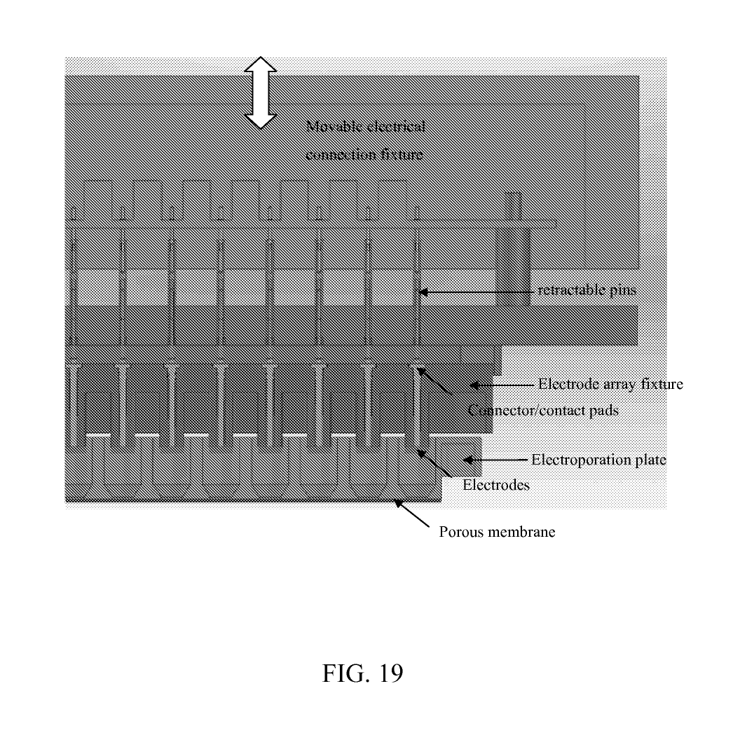

FIG. 19 shows a design of a cell electroporation device for transfecting cells in microtiter plate format. A movable electrical connection fixture may be provided. The connection fixture may be connected to one, two, or more retractable pins. In some embodiments, the same number of pins may be provided as the number of wells and/or electrodes (e.g., 96 for a 96 well plate). An electrode array fixture may be provided with one, two or more connector pads that may interface with the retractable pins. Electrodes may be provided on the electrode array fixture and may be in contact with the connector/contact pads. An electroporation plate may be positioned below the electrode array fixture, and may include a porous membrane substrate.

In accordance with one aspect of the invention, a method may be provided where the electrical connection fixture may be movable. In some embodiments, it may move in a vertical direction. The electrical connection fixture may move up and down with respect to the electroporation plate. The electroporation plate may remain stationary while the electrical connection fixture moves. Alternatively, the electroporation plate may move up and down with respect to the electrical connection fixture while the electrical connection fixture remains stationary. Alternatively, both the electrical connection fixture and the electroporation plate may move.

The retractable pins may be electrical communication with one or more current source. In some embodiments, the retractable pins may move with the electrical connection fixture. Thus, when the electrical connection fixture moves downward, so may the pins. The pins may be fixed relative to the electrical connection fixture. Alternatively, the pins may move independently of the electrical connection fixture and/or may be extended or retracted relative to the electrical connection fixture. The pins may be extended or retracted in a vertical direction with respect to the electrical connection fixture. The pins may fit within the connector or contact pads of the electrode array fixture. In some embodiments, the connector/contact pads may be formed of an electrically insulating material, and the pins may contact the connector/contact pads, fit within, and/or fit partially or all of the way through. The connector pads may allow the pins therein to be electrically isolated and/or insulated from one another. In some other embodiments, the connector pads may be formed of an electrically conductive material, and the pins may contact the connector pads and/or fit partially within or through the connector pads. The connector pads may be provided in the electrode array fixture and may be surrounded by an insulating material. Thus, the connector pads may be electrically insulated from one another.

The pins may be formed of an electrically conductive material and may be connected to the current sources. In some embodiments, pins may include, but are not limited to, silver, copper, aluminum, gold, platinum, or non-metallic conductors, or any alloys or combinations thereof. One, some, or all of the pins may be electrically connected to the same current source. One, some, or all of the current sources may be connected to the same pin. In some embodiments, a plurality of pins may be connected to a plurality of current sources. The current sources may be independently and individually controllable, or groups of current sources may be controlled. Various electrical properties of the current sources may be controlled, such as, but not limited to, polarity, current duration, pulse width, time between pulses, or amplitude of current. An electrode may be in electrical communication with the pin. The electrode may contact the pin directly, or may contact a connector pad that may be contacting the pin. Thus, the electrodes may be in electrical communication with one or more current source.

The electrodes may move up and down with an electrical connection fixture and/or an electrode array fixture. Alternatively, the electrodes and/or electrode array fixture may remain in a fixed position while the electrical connection fixture may move up and down. The relative positions of an electrodes and an electroporation plate may change relative to one another. For example, the electrodes may move up and down while the electroporation plate remains in a fixed position. The electrodes may remain fixed while the electroporation plate moves up and down. Alternatively, both the electrodes and electroporation plates may be moving. Any discussion herein of movement, including up and down movement, may also be applied to lateral or horizontal movement. For example, the electrodes may also move sideways relative to the electroporation plate, or the movable electrical connection fixture and/or electrode array fixture may move sideways relative to one another or to the electroporation plate.

In some embodiments, the electrode may come into contact with the contents of an electroporation plate. For example, the electroporation plate may include a plurality of chambers configured to confine or contain an electrically conductive liquid. The electrodes may come into electrical contact with the electrically conductive liquid. The electrodes may be brought within the chambers of the electroporation plate. In some embodiments, the electrodes may remain near a top portion of the chambers, or may be brought past the halfway mark of the chambers, or near the bottom of the chambers. The electroporation plate may include a porous membrane. The porous membrane may be provided on the bottom of the plate. In some embodiments, the bottom surface of the chamber may be formed of the porous membrane. The electrodes may or may not be brought into contact with the porous membrane.

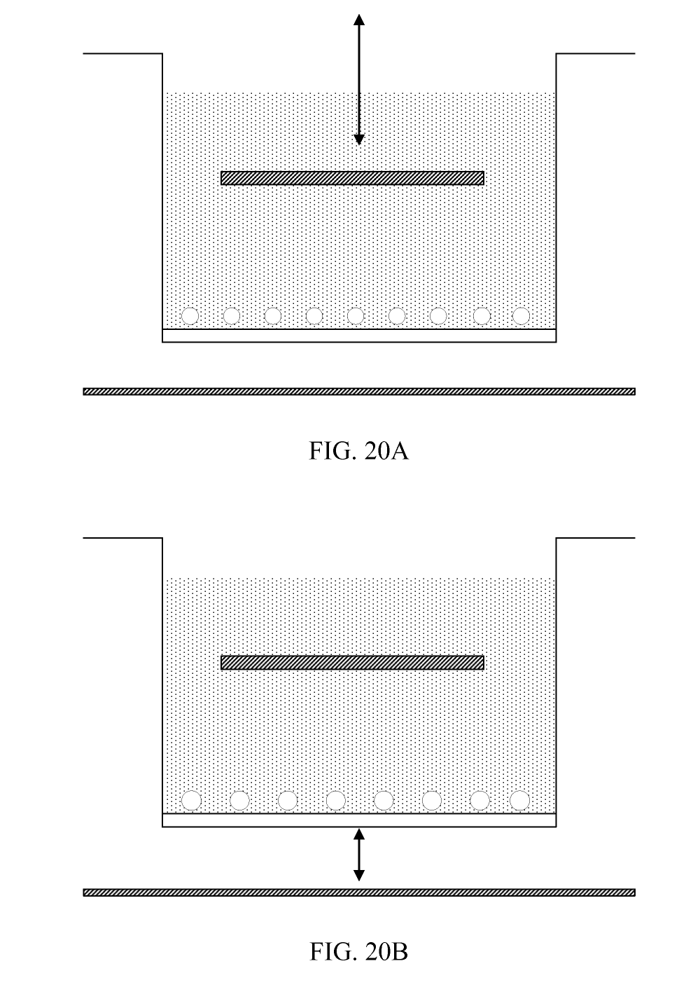

FIG. 20A shows an example where an electrode is brought into electrical communication with contents of a chamber through the top of the chamber. A chamber may include one or more side surfaces and a bottom surface. The chamber may have an opening on the top. In some embodiments, the bottom surface may be formed of a porous membrane. Alternatively, it may be formed of a bottom electrode. A population of host cells may contact a surface of the chamber. In some embodiments, the host cells may contact the bottom surface of the chamber. The chamber may also have a fluid therein. An electrode may be brought into electrical contact with the fluid. In some embodiments, the electrode may be brought into electrical communication with the host cells. The electrode may be brought into contact with the fluid through the opening at the top of the chamber. An electrode may also be selectively removed from contact with the fluid through the opening at the top of the chamber. The electrode may be brought into or taken out of the chamber as desired.

A bottom electrode may be provided. The bottom electrode may be beneath the porous membrane and may or may not contact the porous membrane. A bottom electrode may contact a fluid that may contact the porous membrane. In some embodiments, the bottom electrode may contact an electrically conductive fluid that may contact the porous membrane, thus allowing the bottom electrode to be in electrical communication with the porous membrane and/or host cells.

FIG. 20B shows an example where an electrode is brought into electrical communication with contents of a chamber through the bottom of the chamber. A chamber may include one or more side surfaces and a bottom surface. The chamber may have an opening on the top. In some embodiments, the bottom surface may be formed of a porous membrane. A population of host cells may contact a surface of the chamber. In some embodiments, the host cells may contact the bottom surface of the chamber. The chamber may also have a fluid therein. A top electrode may be within electrical contact with the fluid. The top electrode may be brought into or out of contact with the fluid, or may remain in electrical contact with the fluid. The top electrode may be in electrical communication with the host cells. The top electrode may be in contact with the fluid through an opening at the top of the chamber.

A bottom electrode may be provided. The bottom electrode may be beneath the porous membrane and may or may not contact the porous membrane. A bottom electrode may contact a fluid that may contact the porous membrane. In some embodiments, the bottom electrode may contact an electrically conductive fluid that may contact the porous membrane, thus allowing the bottom electrode to be in electrical communication with the porous membrane and/or host cells. In other embodiments, the bottom electrode may be brought into electrical contact with a conductor that contacts the electrically conductive fluid beneath the porous membrane. The bottom electrode may be brought into up or down into closer proximity to the porous membrane. In some embodiments, the bottom electrode may be brought into or out of electrical communication with the porous membranes and host cells.

A cell transfection device may be provided in accordance with an aspect of the invention. The cell transfection device may include an electrode assembly and a plate holder configured to hold a transfection plate, wherein the plate holder is configured to translate the plate into alignment with the electrode assembly. Preferably, the plate holder and/or the electrode assembly is movable in an upward or downward direction so that the electrode assembly and the transfection plate or contents thereof are brought into contact with one another.

The transfection plate may include a plurality of wells configured to contain a population of host cells. The population of host cells may be formed of a plurality of types of cells. The wells may also be configured to contain an electrically conductive liquid therein. In some embodiments, the wells are open on the top. The electrode assembly may include a plurality of electrodes, wherein the electrodes correspond to the plurality of wells.

In some embodiments, the cell transfection device may have a plate holder which has (1) an open configuration wherein the transfection plate can be loaded outside a housing of the cell transfection device, and (2) a closed configuration wherein the plate holder is within the housing of the cell transfection device. The electrodes may be brought into electrical contact with their corresponding wells through the open tops of the wells. In some instances, the electrode assembly may be adapted to remain substantially stationary during said translation of said plate. Alternatively, the electrode assembly may move during the translation of the plate.

A cell transfection device may be provided in accordance with an aspect of the invention. The cell transfection device may include an electrical connection assembly, and a plate holder configured to hold a transfection assembly, the transfection assembly comprising a transfection plate and an electrode plate, wherein the plate holder is configured to translate the transfection assembly into alignment with the electrical connection assembly. Preferably, the plate holder and/or the electrical connection assembly is movable in an upward or downward direction so that the electrical connection assembly and the transfection assembly or contents thereof are brought into contact with one another. The transfection plate may include a plurality of wells configured to contain a population of host cells. The population of host cells may be formed of a plurality of types of cells. The wells may also be configured to contain an electrically conductive liquid therein. In some embodiments, the wells are open on the top. The electrical connection assembly may include a plurality of electrodes, wherein the electrodes correspond to the plurality of wells. In some embodiments, the cell transfection device may have a plate holder which has (1) an open configuration wherein the transfection assembly can be loaded outside a housing of the cell transfection device, and (2) a closed configuration wherein the plate holder is within the housing of the cell transfection device. The electrodes may be brought into electrical contact with their corresponding wells through the open tops of the wells. In some instances, the electrical connection assembly may be adapted to remain substantially stationary during said translation of said plate. Alternatively, the electrical connection assembly may move during the translation of the plate.

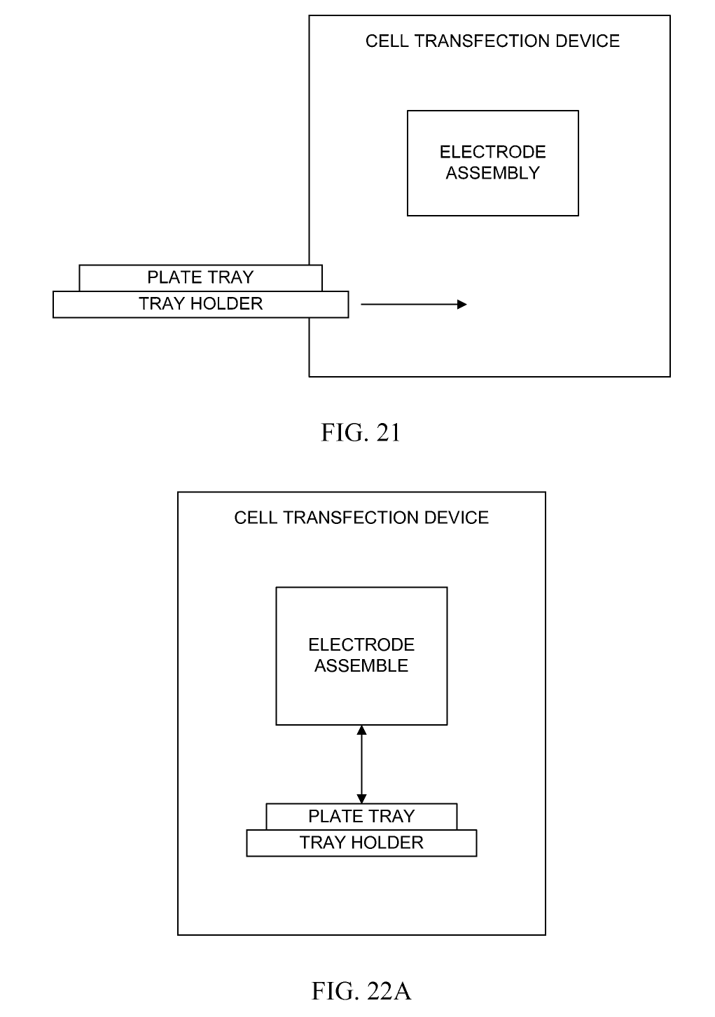

FIG. 21 shows a depiction of a transfection plate being translated into alignment with an electrode assembly. In accordance with an aspect of the invention, a cell transfection device may be provided, which may comprise an electrode assembly and a plate holder configured to hold a transfection plate, wherein the plate holder is configured to translate the plate into alignment with the electrode assembly. The electrode assembly may comprise a plurality of electrodes. In some embodiments, the electrode assembly may include any of the configurations discussed elsewhere herein.

The plate holder may be configured to hold or support the transfection plate and move it into a desired alignment. In some embodiments, the plate holder may include a tray upon which the transfection plate may rest. The plate holder may fixedly hold the transfection plate to the plate holder. For example, the plate holder may have a locking mechanism, which when engaged, the transfection plate may not move relative to the plate holder. Examples of locking mechanisms may include but are not limited to clamps, screws, press fitted components, sliding portions, magnetic components, or ties. In some embodiments, the transfection plate may fit into a slot or shape within the plate holder that may prevent the transfection plate from moving side to side in a horizontal manner, while allowing the plate to be removed vertically. Alternatively, the transfection plate may rest on the plate holder and be able to freely move in a horizontal or vertical manner.

The plate holder may be configured to have a first position where the plate holder accepts the transfection plate, and a second position where the plate holder is aligned with the electrode assembly. In some embodiments, the first position may include the plate holder protruding from a housing of the cell transfection device. The plate holder may be partially or fully protruding from the device housing. For example, a tray may be in an open position so that the tray is protruding from the housing of the device and the top of the tray is exposed outside the device. A transfection plate may be placed on the tray outside the housing of the cell transfection device at this time.

The second position of the plate holder may include the plate holder being fully within the housing of the cell transfection device. This position may also cause a transfection plate to be brought fully within the housing of the device. The plate holder may be translated in a horizontal/lateral direction when moving from the first position to the second position. For example a tray may slide horizontally between a first open position and a second closed position. There may or may not be a vertical component to the movement between the first and second positions. When the plate holder and transfection plate are in the second position, the transfection plate may be may be aligned with an electrode assembly. For example, they may be in vertical alignment. One or more electrode of the electrode assembly may be vertically aligned with one or more chamber of the transfection plate. In some implementations, the plate holder and/or the electrode assembly is movable in an upward or downward direction so that the electrode assembly and the transfection plate are brought into contact with one another.

The cell transfection device may include one or more types of actuators that may enable the plate holder to move between the first and second position, and the relative movement between the electrode assembly and the transfection plate. For example, the cell transfection device may utilize one or more motors, solenoids, linear actuators, pneumatic actuators, hydraulic actuators, electric actuators, piezoelectric actuators, or magnets. The actuators may be in communication with a power source. The power source may be integral to the device or may be external to the device. For example, power sources may include a battery, generator, grid utility, renewable power sources, or any combination thereof.

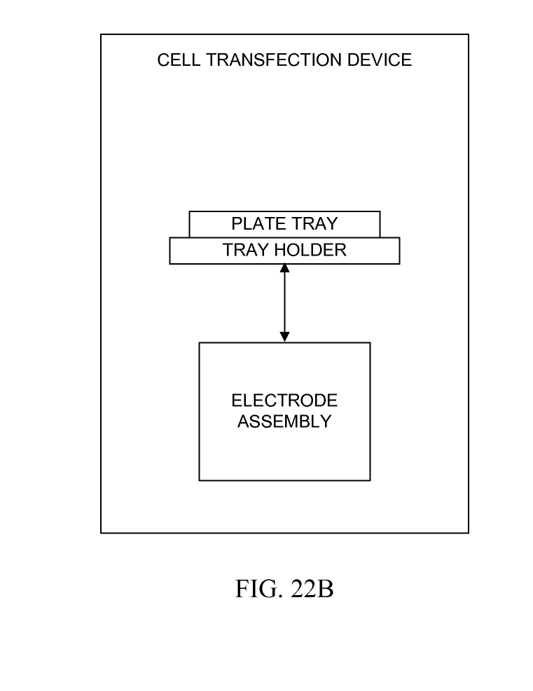

FIG. 22A shows an example of a transfection plate and an electrode assembly being brought into contact with one another. In one example, an electrode assembly may be provided above the transfection plate and/or plate holder. The electrode assembly may be brought down to the transfection plate and/or the transfection plate may be brought up to the electrode assembly. In some embodiments, they may move in a vertical direction without substantially moving in a horizontal direction. Alternatively, there may be a horizontal component to their movement. The transfection plate may or may not be in contact with the plate holder when electrical contact is made with the electrode assembly. The electrode assembly may include a plurality of electrodes that may be brought into electrical contact with a plurality of chambers.

FIG. 22B shows another example of a transfection plate and an electrode assembly being brought into contact with one another. In one example, an electrode assembly may be provided below the transfection plate and/or plate holder. The electrode assembly may be brought up to the transfection plate and/or the transfection plate may be brought down to the electrode assembly. In some embodiments, they may move in a vertical direction without substantially moving in a horizontal direction. Alternatively, there may be a horizontal component to their movement. The transfection plate may or may not be in contact with the plate holder when electrical contact is made with the electrode assembly. For example, a plate tray holder may be withdrawn from the transfection plate when a bottom electrode is brought into contact. The electrode assembly may include a single electrode or a plurality of electrodes that may be brought into electrical contact with a plurality of chambers.

In some instances, electrode assemblies may be provided both above and below the transfection plate. The electrode assemblies and/or transfection plate's positions relative to one another may be varied as described above. Any components, characteristics, features, movements, and steps as described in U.S. Pat. No. 6,677,151 may be incorporated by reference in its entirety.

IX. Device and Interface Connection

In accordance with an embodiment of the invention, a cell transfection apparatus may be provided. A housing may be provided for a cell transfection apparatus. The housing may enclose components of the apparatus. For example the housing may enclose an electrode assembly, and electronics associated with the apparatus. In some embodiments, a cell transfection apparatus may be provided as a single device that is enclosed by the housing. In other embodiments, a cell transfection apparatus may include multiple devices, such as device enclosed by the housing and one or more external device (e.g., a computer, or other device as described elsewhere herein). The cell transfection apparatus may include one or more components that may include hardware for cell electroporation, control software for the electroporation, and an interface therebetween.

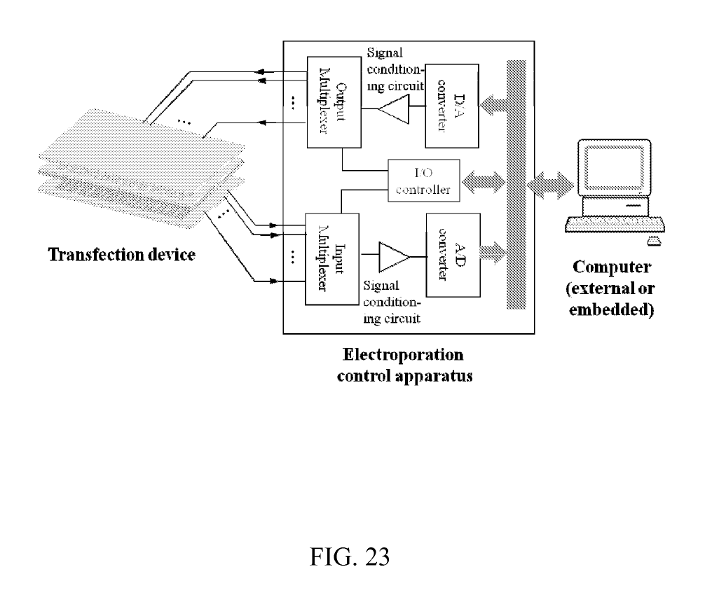

FIG. 23 shows an example of a configuration of a cell transfection apparatus. A cell transfection apparatus may include a transfection device, an electroporation control apparatus, and a computer or processor which may be external or embedded.

A transfection device may include a transfection plate. The transfection device may include additional hardware associated with cell electroporation. For example, the transfection device may include a plate holder which may support the transfection plate. The transfection device may also include an electrode assembly. The electrode assembly may include an electrode array fixture, which may or may not have connector pads. The electrode assembly may also include an electrical connection fixture, which may include pins. The electrical connection fixture may be movable relative to the transfection plate. The pins may or may not be retractable. A transfection device may also include one or more current source in electrical communication with the pins. A transfection device may include any of the plates, devices, or apparatuses as discussed elsewhere herein. The transfection device may be contained within the housing. In some instances, the transfection device may be contained within the housing during a closed position, while parts of the transfection device may be exposed outside of the housing during an open position.

An electroporation control apparatus may provide an electrical interface between the transfection device and the computer/processor. The electroporation control apparatus may include a digital/analog (D/A) converter. The D/A converter may be in electrical communication with a signal conditioning circuit. The signal conditioning circuit may provide an output to an output multiplexer. The output multiplexer may be in electrical communication with the transfection device. The D/A converter may be in electrical communication with an electrical bus, which may provide a digital signal that may be provided to the D/A converter, where it is converted to an analog signal, which may then be conditioned by the signal conditioning circuit, and provided to the output multiplexer, which sends the conditioned signal to the transfection device to effect electroporation and/or measurements.

A transfection device may also be in electrical communication with an input multiplexer of the electroporation control apparatus. The input multiplexer may receive an analog electrical signal from the transfection device. The analog signal may be provided as a result of cell electroporation and/or diagnostics. Measurement of electrical properties of the transfection device may result in an analog signal. The input multiplexer may be in electrical communication with a signal conditioning circuit, which may condition the analog signal. The signal conditioning circuit may be in electrical communication with an analog/digital (A/D) converter, which may receive the conditioned analog signal and convert it to a digital signal. The A/D converter may be in electrical communication with an electrical bus. The same electrical bus may be in communication with the D/A converter and the A/D converter. The electrical bus may be in electrical communication with a power source. The power source may be integral to the cell transfection apparatus or external to the apparatus. The power source may be contained within the housing of the cell transfection or external to the cell transfection apparatus housing. A power source may include a battery, electrical plug connected to an outlet, or any other source of energy.

An electroporation control apparatus may also include an input/output (I/O) controller. The I/O controller may be in communication with an electrical and/or communication bus. In some embodiments, the I/O controller may be in two-way communication with the bus. Alternatively, the communications may be one-way. The I/O controller may also be in communication with an output multiplexer and an input multiplexer. Such communications may be one-way or two-way communications. The I/O controller may receive signal indicative of the electrical signals being provided to the transfection device and/or received from the transfection device. The I/O controller may communicate with the bus to control the electrical signals being provided to the D/A converter. The I/O controller may also be communicating with the bus which may be in electrical communication with the computer/processor. The electroporation control apparatus may be contained within the housing. Alternatively, all or a portion of the electroporation control apparatus may be provided external to the housing.

The computer/processor may be embedded or external to the transfection device. In some embodiments, the computer/processor may have a two-way communication with an electrical and/or communication bus to receive information and/or provide instructions for electroporation and/or diagnosis. The computer may provide instructions to an I/O controller, which may determine the characteristics of electrical currents that are provided to the transfection device. Similarly, the computer may receive feedback from the I/O controller which may include electrical property measurements taken from the transfection device. The computer may have one or more protocol stored therein that may include instructions for effecting electroporation or diagnosis. The computer may also include instructions for opening and/or closing a tray of the device, loading and/or unloading a transfection plate, or effecting the movement of the transfection plate and/or electrode assembly within the device.

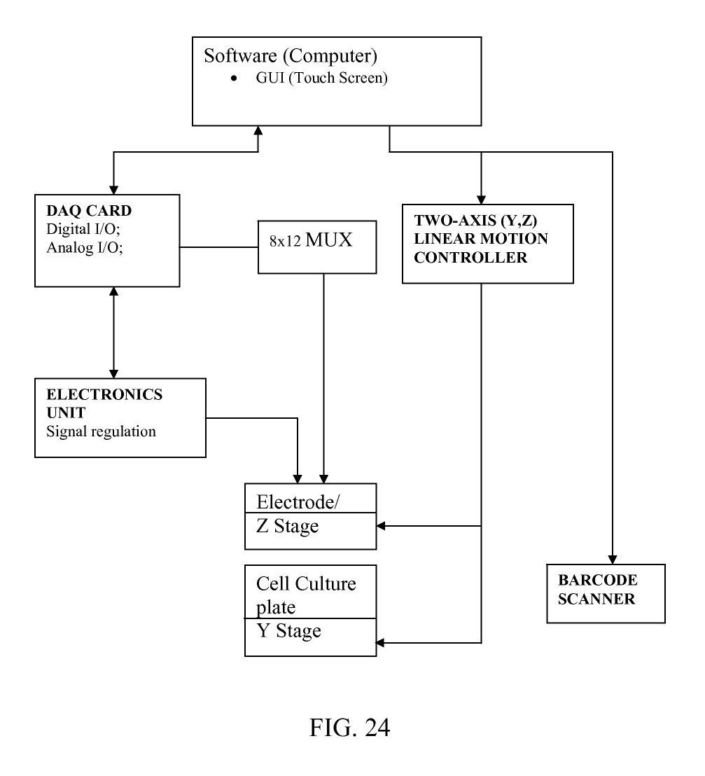

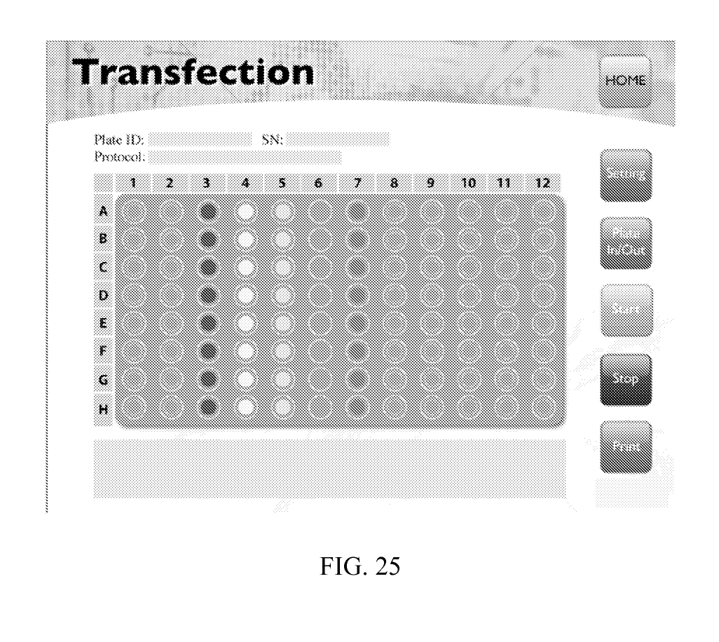

FIG. 24 shows an example of a cell electroporation system in accordance with an embodiment of the invention. A software may be provided on a computer. Tangible computer readable media may be stored on a memory, which may include logic, code, and/or instructions for executing the software. The computer may also include a graphical user interface (GUI). In some embodiments, the GUI may be formed from a touch screen. The software may be in electrical communication with a DAQ card, which may include digital and analog I/O. The DAQ card may be in electrical communication with an electronics unit. The electronics unit may be used for signal regulation. The DAQ card may also be in communication with a multiplexer, such as an 8×12 MUX (e.g., for a 96 well 8×12 plate). The multiplexer may correspond to the transfection device. The regulated signal and the multiplexer may provide signals to an electrode. Such signals may control the characteristics of the current provided to an electrode. Such characteristics may include electrical properties such as voltage, current amplitude, duration, pulse length, cycles, or other properties discussed elsewhere herein.

A software may also be in communication with a motion controller. In some embodiments, the motion controller may be a two-axis (Y,Z) linear motion controller. The motion controller may control the movement of the electrode in the Z direction. In some embodiments, the Z direction may be a vertical direction. The Z direction may be the direction of the electrode relative to a cell culture plate. The motion controller may also control the movement of the cell culture plate. The movement of the cell culture plate may be controlled in the Y direction. In some embodiments, the Y direction may be a horizontal direction. The Y direction may be perpendicular with respect to the Z direction. The Y direction may be the direction of motion for a cell culture plate between when it is loaded into a device and when it is brought into alignment with an electrode. In one example, the software may control the horizontal translation of the cell culture plate from a loading position to an alignment position. The software may control the vertical translation of an electrode to be brought into communication with the cell culture plate.

The software may also be in communication with a barcode scanner. The barcode scanner may be used to scan a cell culture plate. Any discussion herein of a barcode scanner may also apply to any device, including optical, magnetic, electrical, chemical, mechanical devices that may read an identifier. A cell culture plate may include a barcode or other identifier (e.g., optical, magnetic, electrical, chemical, mechanical) that may identify the type of plate and/or contents of the plate. For example, a barcode may identify the host cells provided within the transfection plate. In another example, the barcode may identify the dimensions associated with the cell culture plate. In some instances, the barcode may include suggested protocols for the plate.

The barcode scanner may be integrated into the transfection device. For example, when a cell culture plate is loaded, the barcode scanner may scan the device and collect information related to the cell culture plate. In some embodiments, the barcode scanner may be provided within the housing of a cell electroporation apparatus. In other embodiments, the barcode scanner may be provided external to the transfection device. The information collected from the barcode scanner may affect the signals that are provided through the DAQ card to the electrode.

In some embodiments, a cell culture plate may be automatically scanned when it is loaded into a apparatus, or when it is brought into a closed position. Alternatively, the plate may be manually scanned from outside the apparatus. Scanned information may be sent to the apparatus or to an external device. The scanned information may be compared with stored data to determine a suggested protocol.

X. User Interface

In accordance with an aspect of the invention, a display device may be provided, showing a graphical user interface (GUI). The display device may include a video display. In preferable embodiments, the display device may be provided on a cell transfection device. For example, when on the housing of a cell transfection device, a screen may be provided, which may include the video display. In other embodiments, the display device may include a remote screen, computer, laptop, PDA, ‘smart phone’, mobile phone, or any other device configured to provide a video screen. Thus, a display device may be integral or external to the cell transfection device. Any discussion of a client computer or display device may be applied to any other type of display device. In some embodiments, the display device may include a touchscreen. The user may be able to interact with a graphical representation via touching the screen directly to effect an interaction. In other embodiments, a user may be able to interact with a graphical representation via any other user interactive control, such as a keyboard, a button, a switch, a mouse, a trackball, a stick, or a remote controller.

A user may be a medical professional, a lab technician, a subject, or anyone who may interact with the electroporation device.

A user interface provided in accordance with the invention herein may be displayed on a device, or across a network such as the Internet or a local area network. One example may include a cell transfection device comprising a video display with at least one display page comprising data. The data may include cell transfection data, which may include data relating to the electroporation of host cells, the types of host cells, types of foreign substances introduced to the host cells, the locations of host cells, provided transfection plate information, error detection, post-transfection data, or electroporation protocol management information. Any discussion herein relating to cell transfection data may be applied to other types of data.

Video displays (e.g., display devices) may include devices upon which information may be displayed in a manner perceptible to a user, such as, for example, a computer monitor, cathode ray tube, liquid crystal display, plasma display, light emitting diode display, touchpad or touchscreen display, and/or other means known in the art for emitting a visually perceptible output. Video displays may be electronically connected to cell transfection device or a client computer according to hardware and software known in the art.

In one implementation of the invention, a display page may include a computer file residing in memory which may be transmitted from a server over a network to a cell transfection device and/or client computer, which can store it in memory. Alternatively, the display page may be provided by a computer file already residing in a device, such as a transfection device or client computer. A device may receive computer readable media, which may contain instructions, logic, data, or code that may be stored in persistent or temporary memory of the device, or may somehow affect or initiate action by a device. One or more programs or algorithms may be provided on a device, and may be executable by the device. Similarly, one or more servers may communicate with one or more devices across a network, and may transmit computer files residing in memory. For example, the computer files may include protocols that may be imported to or from a device. The network, for example, can include the Internet or any network for connecting one or more devices to one or more servers.

At a client computer, the display page may be interpreted by software residing in memory of the client computer, causing the computer file to be displayed on a video display in a manner perceivable by a user. The display pages described herein may be created using a software language known in the art such as, for example, the hypertext mark up language (“HTML”), the dynamic hypertext mark up language (“DHTML”), the extensible hypertext mark up language (“XHTML”), the extensible mark up language (“XML”), or another software language that may be used to create a computer file displayable on a video display in a manner perceivable by a user. Any computer readable media with logic, code, data, instructions, may be used to implement any software or steps or methodology. Where a network comprises the Internet, a display page may comprise a webpage of a type known in the art, or any other graphical user display. A display page may be provided through a wired connection or wirelessly.

A display page according to the invention may include embedded functions comprising software programs stored on a memory device, such as, for example, VBScript routines, JScript routines, JavaScript routines, Java applets, ActiveX components, ASP.NET, AJAX, Flash applets, Silverlight applets, or AIR routines.

A display page may comprise well known features of graphical user interface technology, such as, for example, frames, windows, scroll bars, buttons, icons, and hyperlinks, and well known features such as a touch screen interface or a “point and click” interface. Touching, or pointing to and clicking on a graphical user interface button, icon, menu option, or hyperlink also is known as “selecting” the button, option, or hyperlink. A display page according to the invention also may incorporate multimedia features.

A user interface may be displayed on a video display and/or display page. A cell transfection device or an external device may have access to cell electroporation data. A user interface may be used to display or provide access to cell electroporation data.

The graphical user interface may include a visual representation of a cell transfection plate having a plurality of chambers, wherein the visual representation includes images corresponding to the plurality of chambers. The graphical user interface may also include a user interactive control that may permit a user to define a property relating to at least one chamber via the visual representation.

The display device may include an interactive control that may a user to define a property, where the defined property is further processed to effect a transfection condition based on said defined property. The user may be able to interact with the user interactive control, which may be a touch screen, a keyboard, a button, a switch, a mouse, a trackball, a stick, or remote controller. The transfection condition may be effected in real time. Preferably, the user interactive control permits a user to select at least one chamber for which to define the property.

FIG. 25 shows an example of a touch-screen based GUI for inducing cell electroporation in a 96-well format in accordance with an embodiment of the invention. The cell electroporation may or may not be SACE. The GUI shows an example of a visual representation of the 96-well transfection plate. For example, the visual representation may show a depiction of a top view of a transfection plate. For example, if the transfection plate has n number of wells, the visual representation may show the n wells, and they may be configured to visually correspond to how they are configured in the transfection plate. In some embodiments, a transfection plate may have an array of wells, with a plurality of rows and a plurality of columns. The visual representation may depict a corresponding array of wells, with the same number of rows and columns as the transfection plate. The visual representation may be provided for any of the plate formats as described elsewhere herein.

In some embodiments, the visual representation may show a graphical representation of the transfection plate. The visual dimensions of the representation may or may not correspond to the dimensions of the plate and/or the ratio of dimensions of the plate. In some embodiments, the transfection plate may be represented by a rectangular shape. Similarly, the visual representation may show representations of the wells of the transfection plate. These well representations may or may not have the same size, proportions, or shape as the wells of the transfection plate. In some embodiments, the wells may be represented by circular shapes. The circular shapes may be provided in a plurality of rows and columns within the visual representation of the transfection plate.

In some embodiments, the various visual representations of the wells may be visually emphasized. For example, the well representations may be filled in with a different color, or may be bolded, highlighted, or a different size compared to other wells. In some instances, different visual emphasis may be provided to represent different information associated with the wells. For example, if columns of different colored wells are shown, this may indicate that the different wells are undergoing different protocols, that some wells may contain different host cells, or that some wells are empty.

The visual representation may also include one or more label corresponding to a location of a well. For example, labels may be provided corresponding to rows of wells. For example, each row may be labeled with a letter (e.g, A-H as shown). Labels may also be provided corresponding to columns of wells. For example, each column may be labeled with a number (e.g., 1-12 as shown). Each label may be visually mapped to its corresponding well or set of wells (e.g., provided in the same row or column as the corresponding wells). In some embodiments, the physical transfection plate may include corresponding labels.

The visual representation may also include one or more user interactive control that may define a property relating to a well in the visual representation. For example, the screen may show buttons, which may be accessed via touchscreen or any other techniques described. Some examples may include controls for setting, plate in/out, start, stop, and print. The user may also be able to directly contact a well representation. In some embodiments, fields may be provided that may allow a user to enter information associated with the transfection plate. For example, a user may enter a plate ID, serial number, experiment ID, or protocol.

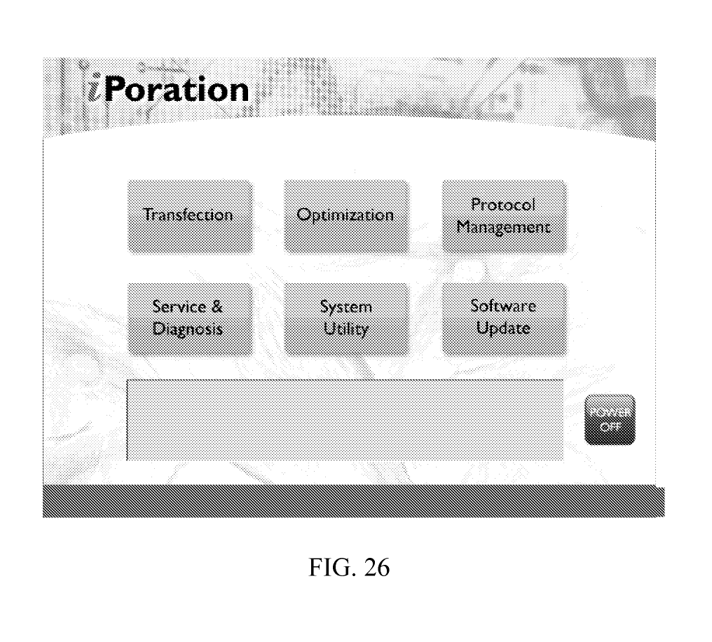

FIG. 26 shows an example of a user interface display providing main options to a user. When a user accesses a display device, an initial menu may be displayed to the user. Options such as transfection, optimization, protocol management, service & diagnosis, system utility, and software update may be displayed to the user. A cell transfection device may be able to perform one or more options, which may include but is not limited to cell transfection, or other options mentioned. The user may also be presented with an option to turn the device power off.

In some embodiments, the various options may be displayed in any visual manner to the user. For example, the options may be displayed as buttons that may be arranged in one or more row and/or column. A user may touch the button via touchscreen to select the option. A user may select an option in any other manner discussed elsewhere herein. This may direct a user to a new screen or display.

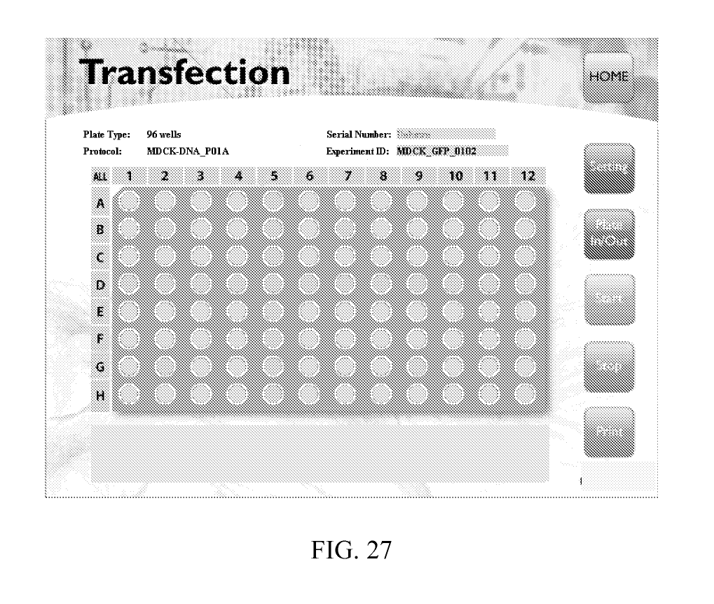

FIG. 27 shows a user interface used for transfection. For example, a user may select a transfection option from a main menu and be directed to the user interface for cell transfection. The transfection display may include a visual representation of a transfection plate with wells. A user may select any combination of wells to transfect. For example, a user may select individual wells to transfect. Or a user may be able to select an entire column, row, or group of wells. For example, selecting a label for a column or row may cause the entire column or row to be selected. Selecting an option for “ALL” may cause all of the wells to be selected. The selected wells may be highlighted or visually emphasized in any other manner. This may advantageously provide a user with an intuitive interactive display with a great degree of customization for an electroporation process.

The selected wells may be associated with a transfection protocol. In some embodiments, one protocol may be applied per transfection plate. Alternatively, multiple protocols may be applied to a transfection plate.

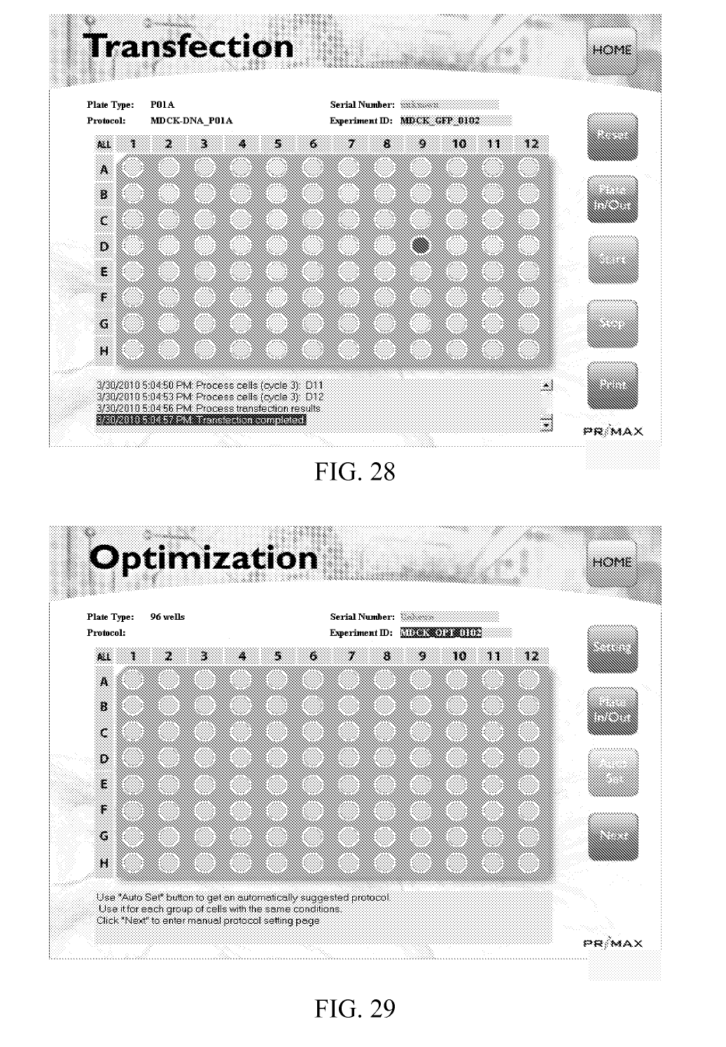

FIG. 28 shows a user interface displaying a transfection status after electroporation. In some embodiments, after electroporation, the various well representations may be presented with different colors or visually emphasized in other ways. These colors may indicate a status of the well after electroporation. For example, a green well may indicate a successfully electroporated well, a yellow well may indicate a well that has questionably been electroporated well, and a red well may indicated a failed well. Other statuses or visual indicators may be used for the same or other well statuses. This may advantageously provide the user with a quick view on the results of an electroporation process.

In some embodiments, the well representations may display the well status only after transfection has been completed. Alternatively, the well representations may display the well status before transfection has been completed as well. For example, while a particular transfection cycle is occurring a well representation may have a particular color. Or while a certain percentage of transfection cycle has occurred, the well representation may have a particular color. In another example, the well representations may have a particular color while transfection results are being processed.

In some embodiments, a comments or status section may be provided. During a transfection process, the current status of the cells may be indicated. For example, a time may be provided along with a description of whether cells are being processed, which cells are being processed, which cycle is being used, whether transfection results are being processed, or whether transfection is completed. Such description may be provided in written or visual form. For example, a visual bar may be provided to show the status of the status of the transfection process relative to completion.

FIG. 29 shows an example of an optimization graphical user interface. An optimization graphical user interface may show a representation of a transfection plate with wells. In some embodiments, user interactive controls may be provided which may include controls for settings, plate in/out, autoset, and next. For example, selecting an option for auto set may cause the program to offer an automatically suggested protocol. Selecting an option for next may direct a user to a manual protocol setting page.

In some embodiments, a user may select an individual well, a group of wells, or all wells in order to provide a protocol. For example, a user may make a well selection from the visual representation of wells (where the well selection may be for an individual well, group of wells, or all wells). The well selection may include one or more wells with the same condition. For example, a user may select a group of wells with the same condition and select an option for auto set, which may offer an automatically suggested protocol. In another example, a user may select an individual well and select an option for next, which may direct the user to a manual protocol setting page. In some embodiments, directions or options may be displayed on the graphical user interface. For example, directions about auto set or next options may be provided.

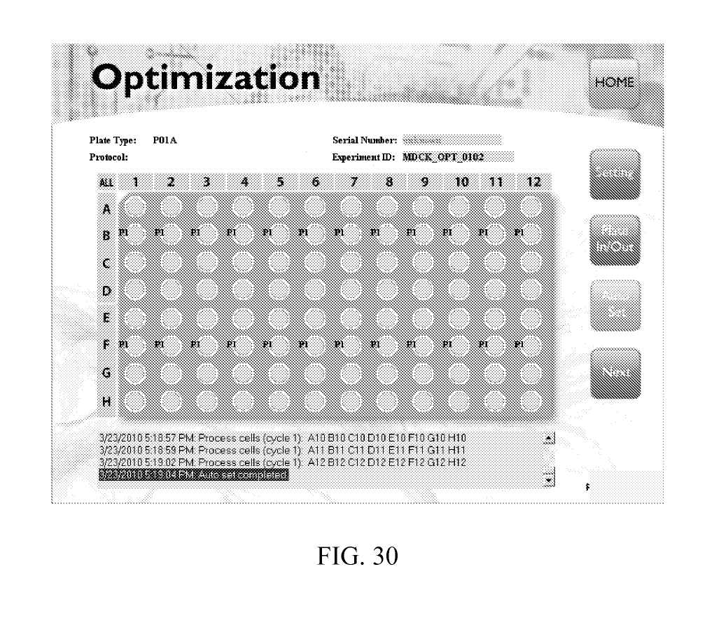

FIG. 30 shows a system suggested protocol based on a detected electrical property of a cell layer. For example, if a user selects an auto set function, a graphical user interface, as shown in FIG. 30 may be provided. The suggested protocol may be provided based on a detected electrical property of a cell layer within the wells. In another example, the suggested protocol may be provided based on information entered by the user about the cells within the wells. In some embodiments, the electrical properties may only be detected for wells that have been selected by a user for the auto set option. Alternatively, the electrical properties of all cells or groups of cells may be measured.

The selected protocol may provide improved, increased, or optimized electroporation of cells. The detected electrical properties and/or any information about the transfection apparatus (e.g., collected from a barcode scanner) may cause a protocol to be selected that may provide favorable cell electroporation conditions.

The suggested protocol may be provided in a visual manner. For example, a visual representation of a transfection plate and visual representation of wells may have a suggested protocol visually mapped to the corresponding wells. For example, if the program suggests a protocol P1 for a group of wells, the P1 may be displayed in a manner to visually correspond the P1 with the associated wells. For example, the P1 may be displayed in close proximity to the corresponding well (or rows, columns, or groups of wells), adjacent to the corresponding well (e.g., horizontally or vertically adjacent to the corresponding well), overlapping the corresponding well, within the corresponding well, with a pointer or line connecting the protocol and corresponding well, or associating the protocol with a key that is visually incorporated with the corresponding well (e.g., color).

In some embodiments, the suggested protocol may be provided just for user selected wells. Alternatively, protocols may be suggested for a group of wells, or all wells within the transfection plate. One suggested protocol may be displayed for selected wells. Alternatively, multiple suggested protocols may be provided for selected wells. For example, within a transfection plate, a P1, P2, P3, etc. may be provided.

A status display may be provided, which may indicate to the user the status of the protocol suggestion. For example, a time may be provided along with indicators of whether wells are being processed, which wells are being processed, whether electrical property measurements are being taken, whether the auto set is being processed, or whether the auto set has been completed. In some embodiments, such status updates may be provided in the form of words, or in a visual format.

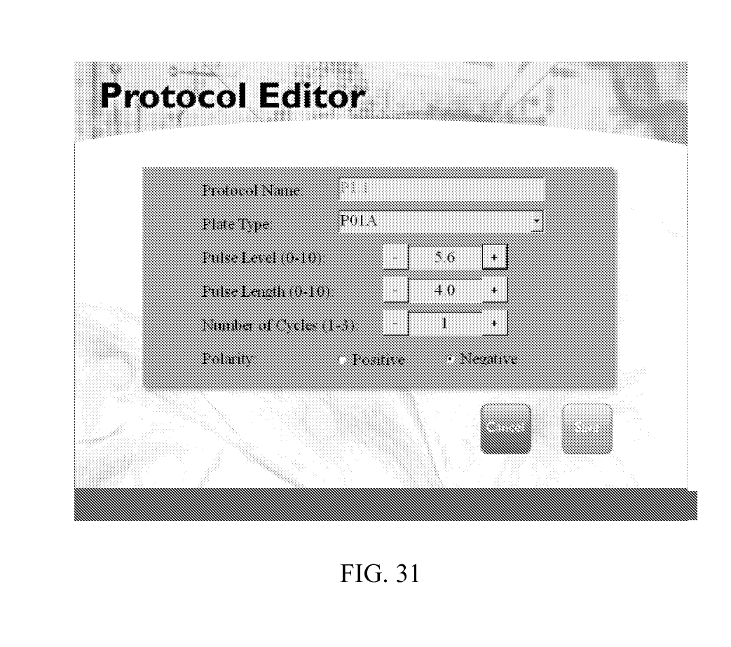

FIG. 31 provides an example of a user interface where a user may further edit a protocol. For example, a user may be provided with a suggested protocol. The user may be able to view parameters or configurations associated with the suggested protocol. The user may be able to edit the parameters or characteristics associated with the suggested protocol. A parameter of a suggested protocol may have a starting value that a user may modify. In some instances, a user may create a new protocol or edit any existing protocol, which may be automatically suggested by a program or manually determined by a user.

In some embodiments, a protocol editing screen may include fields or options for a protocol name, plate type, pulse level, pulse length, number of cycles, or polarity. In some instances, some of the parameter fields may or may not be editable. For example, a protocol name may be fixed while a plate type may be editable. Depending on the parameter type, different user interactive interfaces may be provided. For example, a protocol name may be provided in a text field. A plate type may be selected from a drop down menu. Pulse level, pulse length, or number of cycles may be provided in a text field and/or modified by selecting an increase (+) or decrease (−) button. A polarity may be suggested by clicking on one of multiple options (e.g., positive, negative). Any type of user interactive interface, including but not limited to, text fields, drop down menus, buttons, check boxes, radio buttons, sliding bars, or click and drag interfaces, may be incorporated with any parameter field.

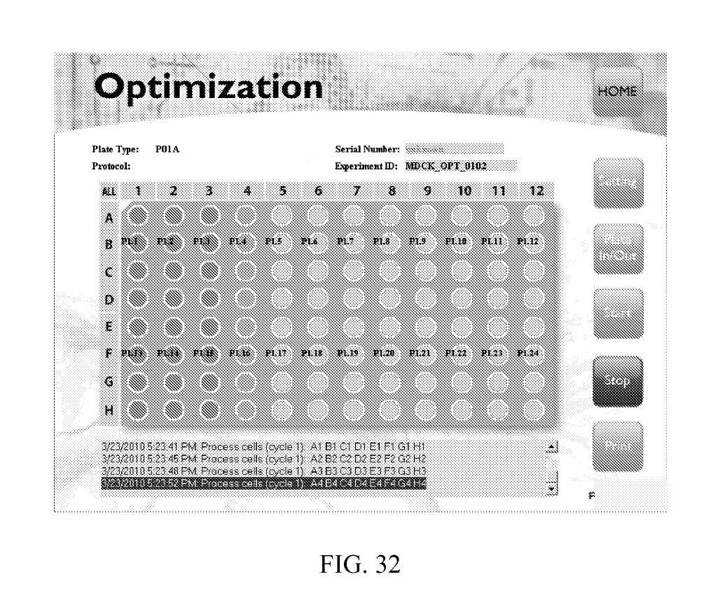

FIG. 32 shows how a plurality of different protocols can be applied to one plate. For example, if 24 wells are selected, up to 24 different protocols may be provided to those selected wells. In some embodiments, a maximum number of protocols may be provided for an entire plate. For example, 1, 2, 4, 8, 15, 24, 30, 40, 50, 60, 70, 80, 90, 100 or fewer protocols may be provided for the entire plate. In some embodiments, the maximum number of protocols may be the number of wells within the transfection plate. Alternatively, no maximum number of protocols may be provided.

In some embodiments, each of the different protocols may be labeled. For example, P1.1, P1.2, P1.3, etc. may be provided. The various protocols may be labeled with any name desired. The protocol names may be an alphanumeric string, or may include symbols.

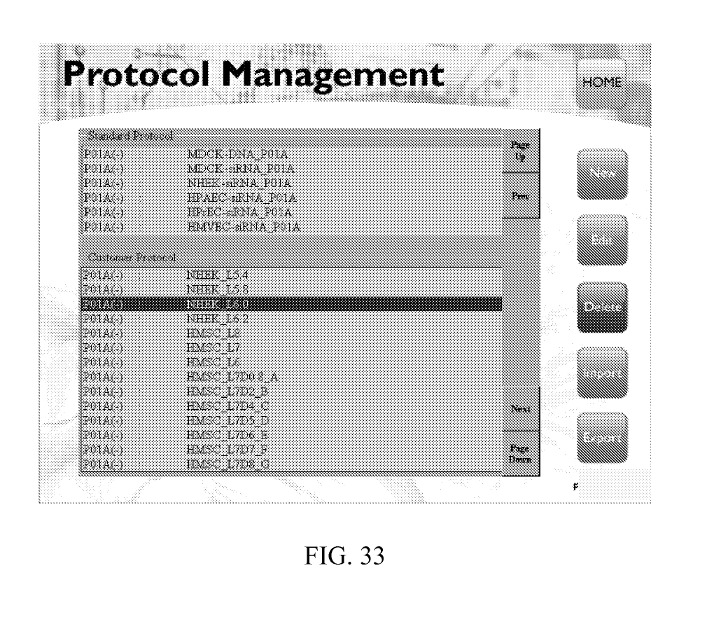

FIG. 33 shows a user interface which allows users to manage their own protocols. In some embodiments, one or more standard protocols may be provided for a cell transfection device. One or more user protocol may be provided as well. A standard protocol may be provided for a transfection device. Such protocols may be stored in a program or algorithm of the device. Such protocols may be provided by a manufacturer or a cell transfection device provider. A standard protocol may or may not be editable by a user. A standard protocol may or may not be renamed by a user. A user protocol may be created and/or edited by a user. A user may be able to create and save protocols for a cell transfection device. In some embodiments, a user may be able to create and save a limited number of protocols. Alternatively, there may be no limit to the number of protocols a user may create or save. A user may be able to edit the user protocols. The user may be able to name the protocols that the user has created.

The various protocols may be displayed on a graphical user interface. The standard protocols may be displayed separately from the user protocols or may be displayed together. In one example, the names of the protocols may be listed. A selected protocol may be highlighted, or any other visual indicator may be used to emphasize a selected protocol. In some instances, only one protocol may be selected at a time. Alternatively, multiple protocols may be selected.

A user may be able to select a protocol through a touchscreen. For example, the user may be able to select the name of a protocol by touching the name of the protocol. A user may also be able to select the name of a protocol via using a page up/page down and/or previous/next button to allow a user to scroll through the list of protocol names.

One or more options may be provided for a user to interact with a protocol. For example, a user may be presented with an option to create a new protocol, edit an existing protocol, delete a protocol, important a protocol, or export a protocol. In some instances, a protocol may be imported from or exported to an external device. The cell transfection device may be wired or wirelessly communicating with the external device.

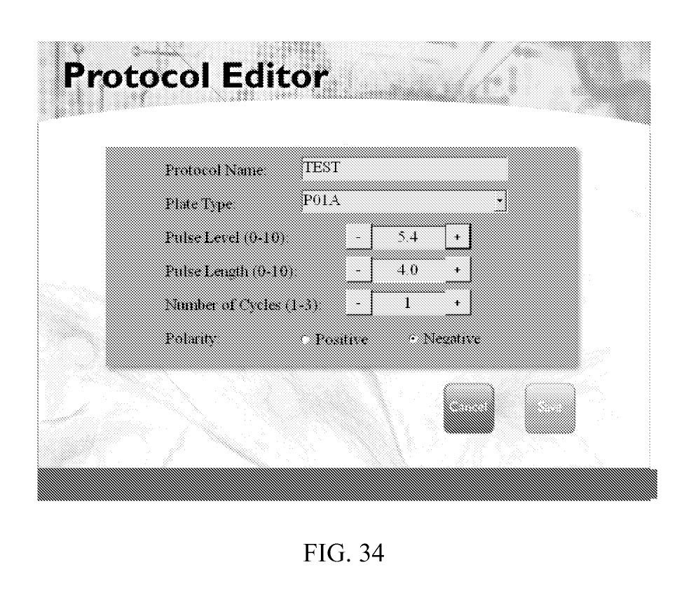

FIG. 34 shows a screen to allow a user to create or edit existing protocols. For example, a user may be able to provide a protocol name, plate type, pulse level, pulse length, number of cycles, or polarity. A plate type may indicate a type of plate used for transfection. For example, a cell transfection device may be able to accommodate multiple different types of transfection plates. Such transfection plates may have different numbers of wells and/or dimensions (e.g., width, length, height, well depth, well width, well length, well spacing, well volume). Such transfection plates may also be used for different types of transfection.

The pulse level may indicate an amplitude of an electric current provided to the contents of a well. In some instances, a range may be provided indicating a pulse level. For example, the pulse level may fall within a numerical range. A user may be able to select a value within the numerical range (e.g., 1-10). In some instances, a preferable current amplitude range may fall within 0.1 to 10. A user may be able to select a discrete value or from along a continuous spectrum. A pulse length may indicate the amount of time that an electrical pulse is provided for. For example, the pulse length may fall within a numerical range. A user may be able to select a value within the numerical range (e.g., 1-10). In some instances, a preferable pulse length range may fall within 0.1-10. A user may be able to select a discrete value or from along a continuous spectrum. The number of cycles may indicate the number of electrical pulses that may occur for a cell transfection process. For example, the number of cycles may fall within a numerical range. A user may be able to select a value within the numerical range (e.g., 1-3). In some instances, a preferable number of cycles may fall within 1-3, or alternatively 1-5, 1-8, 1-10, or 1-20. A user may be able to select a discrete value. The polarity may indicate a positive or negative polarity. This may control the direction that foreign material may be driven into a host cell. This may depend on the polarity of the host cell and/or foreign material.

A user may be able to select an option to save the settings for the protocol. Similarly, the user may be able to cancel a new protocol or changes to an existing protocol.

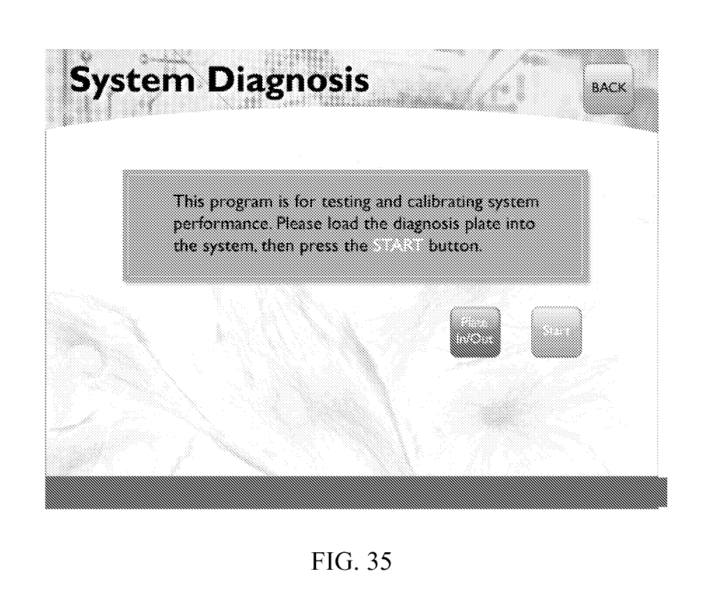

FIG. 35 shows a screen where the system can diagnose whether it is working properly. The diagnostic procedure may test and calibrate system performance. A diagnosis plate may be loaded into a cell transfection device. A plate in/out user interactive control may be provided in the graphical user interface. After a plate is loaded, a start control may be provided to start the diagnostic process. A diagnostic process may include measuring one or more electrical property for one or more well of the diagnosis plate. For example, if a very high resistance is measured (e.g., if a bubble is provided beneath a well as previously discussed), an error may be indicated.

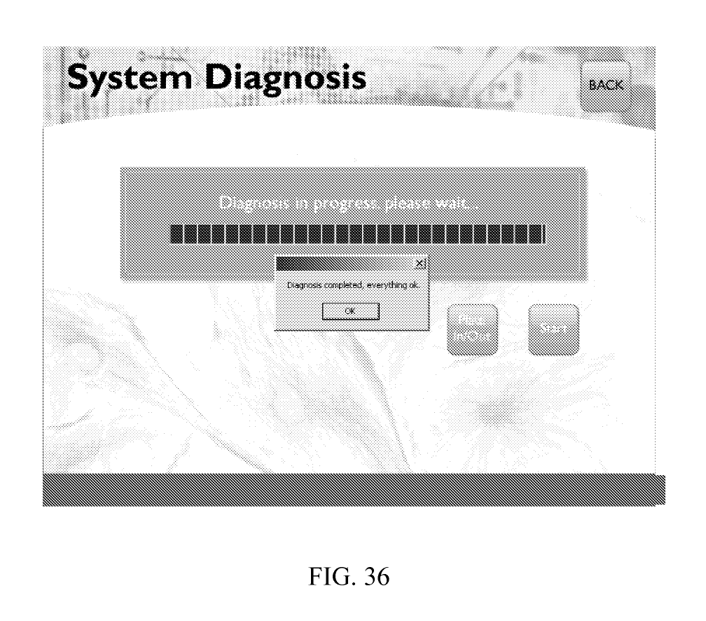

FIG. 36 shows a display after the diagnostics have been completed. During the diagnostic procedure, a status bar or other visual indicator may be provided to show the progress made. For example, a status bar indicating that diagnosis is in progress, showing progress made toward the end of the process. After the diagnosis is completed, a result may be provided to a user. For example, if no errors are detected, an indicator may be provided which may indicate that the diagnosis is complete and everything is ok. If an error is detected, an indicator may be provided that the diagnosis is complete and an error has been detected. It may indicate which error has been detected.

### EXAMPLES

### Example 1

### Electroporation and Transfection of Adherent Cells in Wells

Cells to be transfected are first seeded into a 96-well plate of the present invention. Seeding density can range from 50,000 to 500,000 cells per cm². Cells can include engineered cell lines (such as HepG2, MDCK, and HEK293), primary cells (such as HMEC, HUVEC, and PrEC), or other cell varieties. Cells are then cultured in suitable media under conditions to promote cell adherence and growth, such as an incubator at approximately 37° C. with approximately 5% CO₂. Cells are incubated for a time sufficient to reach a degree of confluence suitable for efficient transfection, such as one to two days, reaching a cell monolayer of greater than 80% confluency.

A solution of plasmid DNA encoding green fluorescent protein (GFP) with a concentration of 10-50 ug/mL is added to each well. Electrical pulses with widths between 100 msec and 5 sec are applied across electrodes above and below the wells. Depending on the cell type and degree of confluence, the amplitudes of the electrical pulses range from 1V to 3V. With a negative electrode above a well and a positive electrode below a well, the naturally negatively charged plasmid DNA is driven toward the well bottom, through any cell located there between, for multiple wells simultaneously. Thus, charged molecules may be actively driven into a cell, in addition to forces of natural diffusion into pores created by the electrical pulses. Using this method to transfect PrEC cells (prostate epithelial cells) is observed to yield greater than 70% transfection, determined by GFP fluorescence.

The above procedure can accommodate transfection of cells with the same or different transfectable material in each well. Alternatively, where the cells are adhered to a porous membrane above the lower electrode, it is possible to introduce charged, transfectable materials, such as nucleic acids, to a fluid space between the well bottom and lower electrode. By properly orienting the electrical field, the transfectable material can be driven up into cells from beneath each of the wells. Where the space below the wells is interconnected, uniformity in transfection across multiple wells is increased by exposing each well to the same transfection material solution.

### Example 2

### Gene Silencing by Transfection of siRNA by Electroporation

Monolayers of adherent PrEC cells in a 96-well plate of the present invention are prepared and transfected with a GFP plasmid vector as in Example 1. Wells are divided into three groups. Group 1 receives no siRNA, group 2 receives siRNA but no exposure to electrical pulses, and group 3 receives both siRNA and electrical pulses. siRNA targeting GFP is added to each well of groups 2 and 3 to a final concentration of 10 nM. By selecting which wells to electroporate using a device of the present invention, wells of groups 1 and 3 are simultaneously electroporated as in Example 1, while wells of group 2 are not. Groups 1 and 2 serve as controls for GFP expression level in the absence of siRNA transfection, and silencing is measured for cells in wells of group 3 relative to those of the controls. Effective silencing can be achieved using these low siRNA concentrations, which are 1 to 3 orders of magnitude lower than concentrations utilized by other systems and methods.

### Example 3

### Well-Region Restricted Transfection by Electroporation

A 96-well plate of the present invention, having a porous membrane serving as the bottom well surface, is treated on the exterior with a UV-curable transparent paste to block the pores in edge region of the membrane in each well. Cells are grown in the interior of the wells as in the above examples. PrEC cells are transfected with a GFP plasmid vector as above. Examination of fluorescent cells reveals efficient transfection of cells only above the region of the membrane in each well having unblocked pores. Other patterns of blocked and unblocked pores within a well can be created to examine relationships between cells having a characteristic arising from a transfected material and non-transfected cells.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

1. A cell transfection apparatus comprising:
(a) a plate having a plurality of chambers, each configured to receive and confine a population of host cells;
(b) a plurality of electrodes, each of which being configured to be in electrical communication with a corresponding chamber of said plate;
(c) an input unit adapted to receive input of one or more transfection parameters from a user; and
(d) a controller for processing said input and effecting an electrical current for a defined period of time to at least one electrode of said plurality, based on the input of said one or more transfection parameters from said user.

2. The cell apparatus of claim 1, wherein the one or more transfection parameters is selected from the group consisting of cell type, membrane resistance, voltage, duration of current flow, rate of current flow, amplitude of current flow, number of electric pulses, shape of electrical pulses, and choice of transfection plate.

3. The cell apparatus of claim 2, wherein the choice of transfection plate is selected from the group consisting of cell attachment substratum, choice of porous membrane, and size of chamber.

4. (canceled)

5. The cell apparatus of claim 1, wherein two way communication is provided between the input unit and controller.

6. (canceled)

7. (canceled)

8. (canceled)

9. (canceled)

10. (canceled)

11. The cell apparatus of claim 1, further comprising an electrode positioned beneath the plate.

12. The cell apparatus of claim 1, wherein the plurality of chambers are arranged in an array comprising one or more row and one or more column.

13. A method for transfecting a population of host cells, comprising:
(a) providing a cell transfection apparatus of claim 1;
(b) receiving input of one or more transfection parameters from a user; and
(c) processing said input and effecting an electrical current for a defined period of time to said population of host cells, based on the input of said one or more transfection parameters from said user.

14. The method of claim 13 further comprising providing a foreign substance to the cell transfection apparatus.

15. The method of claim 14 wherein the foreign substance is selected from the group consisting of DNA, RNA, siRNA, microRNA, peptide, protein, small molecules, or nanoparticles.

16. A method for transfecting a population of host cells, comprising:
providing a cell transfection apparatus of claim 1, wherein the apparatus comprises a touch screen having a visual representation of the plate having a plurality of chambers;
receiving a user input through the touch screen, wherein the input defines a transfection parameter relating to at least one chamber of said plate; and
processing said input and effecting an electrical current for a defined period of time to said chamber, based on the input of said transfection parameter from said user.

17. A method of selecting a transfection condition, comprising:
(a) providing a plate having a plurality of chambers, wherein at least one individual chamber of said plurality contains a population of host cells;
(b) providing a plurality of electrodes, each of which being configured to be in electrical communication with a corresponding chamber of said plate; and
(c) measuring electrical resistance of the contents of said at least one individual chamber and determining a desired transfection condition to effect said transfection.

18. The method of claim 17 wherein the transfection condition comprises a condition selected from the group consisting of duration of voltage, amplitude of voltage, duration of current flow, amplitude of current flow, number of electric pulses, and shape of electrical pulses.

19. The method of claim 17 wherein the plurality of chambers are arranged as an array.

20. The method of claim 19 wherein the plate has a 6, 24, 48, 96, 384, or 1536 well format.

21. The method of claim 17 wherein the host cells are selected from the group consisting of: cell lines, engineered cells, and primary cells.

22. The method of claim 17 wherein the at least one individual chamber contains a population of host cells with density falling between 500 and 1,000,000 cells per square centimeter.

23. (canceled)

24. (canceled)

25. (canceled)

26. (canceled)

27. (canceled)

28. (canceled)

29. (canceled)

30. (canceled)

31. (canceled)

32. A cell transfection device comprising:
an electrical connection assembly; and
a plate holder configured to hold a transfection assembly, the transfection assembly comprising a transfection plate, wherein the plate holder is configured to translate the transfection assembly into alignment with the electrical connection assembly;
wherein the plate holder and/or the electrical connection assembly is movable in an upward or downward direction so that the electrical connection assembly and the transfection assembly or contents thereof are brought into contact with one another.

33. The cell transfection device of claim 32 wherein the transfection plate includes a plurality of wells configured to contain a population of host cells.

34. The cell transfection device of claim 33 wherein the population of host cells are formed of a plurality of types of cells.

35. The cell transfection device of claim 33 wherein the wells are also configured to contain an electrically conductive liquid therein.

36. The cell transfection device of claim 33 wherein the wells are open on the top.

37. The cell transfection device of claim 33 wherein the electrical connection assembly includes a plurality of electrodes, wherein the electrodes correspond to the plurality of wells

38. The cell transfection device of claim 32 wherein the plate holder has (1) an open configuration wherein the transfection assembly can be loaded outside a housing of the cell transfection device, and (2) a closed configuration wherein the plate holder is within the housing of the cell transfection device.

39. The cell transfection device of claim 36 wherein the electrodes are brought into electrical contact with their corresponding wells through open tops of the wells.

40. The cell transfection device of claim 32 wherein the electrical connection assembly is adapted to remain substantially stationary during said translation of said plate.

41. A display device showing a graphical user interface comprising:
a visual representation of a cell transfection plate having a plurality of chambers, wherein said visual representation includes images corresponding to said plurality of chambers; and
a user interactive control that permits a user to define a property relating to at least one chamber via the visual representation.

42. (canceled)

43. (canceled)

44. (canceled)

45. (canceled)

46. (canceled)

47. A method for transfecting a foreign substance into a population of host cells, comprising:
(i) providing a cell transfection apparatus comprising:
(a) a plate comprising a plurality of chambers, each of said plurality of chambers being configured to receive and confine a population of host cells; and
(b) a plurality of electrodes, each of which being configured to be in electrical communication with a corresponding chamber of said plate;
(ii) directing a predetermined electrical current through at least said corresponding chamber to effect transfection of said host cells.

48. The method of claim 47, wherein the predetermined electrical current is directed without controlling voltage across the chamber.

49. The method of claim 47, wherein said transfection results in an uptake of a larger effective quantity of said foreign substance by said population of host cells than the uptake by said host cells via a transfection method utilizing predetermined voltage.

50. The method of claim 47, wherein membranes of said host cells are kept open continuously for at least one millisecond during said transfection.

51. The method of claim 47, wherein the cell transfection apparatus further comprises at least two sets of electrodes, wherein one of the sets is aligned on the top of the plate, and another set is aligned at the bottom of the plate, and wherein the two sets are individually controlled to provide a unidirectional current flowing from the top of the plate to the bottom of the plate or vice versa.

52. The method of claim 32, wherein said transfection assembly further comprises an electrode plate.
